(19) United States
(12) Patent Application Publication
(10) Pub. No.: US 2022/0033739 A1
(43) Pub. Date: Feb. 3, 2022

(54) CLEANING COMPOSITIONS AND USES THEREOF

(71) Applicant: Novozymes A/S, Bagsvaerd (DK)

(72) Inventors: Klaus Gori, Dyssegaard (DK); Lorena Gonzalez Palmen, Akarp (SE); Jesper Salomon, Holte (DK); Kenneth Jensen, Oelsted (DK)

(73) Assignee: Novozymes A/S, Bagsvaerd (DK)

(21) Appl. No.: 17/276,555

(22) PCT Filed: Oct. 9, 2019
(86) PCT No.: PCT/EP2019/077277

§ 371 Date: Mar. 16, 2021

(30) Foreign Application Priority Data
Oct. 11, 2018 (EP) 18199828.7

(51) Int. Cl. C11D 3/386; C11D 11/00

(52) U.S. Cl. CPC C11D 3/38636; C11D 11/0017

(57) ABSTRACT

The present invention relates to compositions such as cleaning compositions comprising a mix of enzymes. The invention further relates, use of compositions comprising such enzymes in cleaning processes.

### REFERENCE TO A SEQUENCE LISTING

This application contains a Sequence Listing in computer readable form, which is incorporated herein by reference.

### BACKGROUND OF THE INVENTION

The present invention relates to compositions such as cleaning compositions comprising a mix of enzymes. The invention further relates to the use of compositions comprising such enzymes in cleaning processes and/or for deep cleaning of organic stains and to methods for removal or reduction of components of organic matter.

### DESCRIPTION OF THE RELATED ART

Enzymes have been used in detergents for decades. Usually a cocktail of various enzymes is added to detergent compositions. The enzyme cocktail often comprises various enzymes, wherein each enzyme targets it specific substrate e.g. amylases are active towards starch stains, proteases on protein stains and so forth. Textile and hard surfaces, such as dishes or the inner space of a laundry machine enduring a number of wash cycles, become soiled with many different types of soiling which may compose of proteins, grease, starch etc. One type of stains may be poly-organic such as stains from body soiling e.g. skin cell debris, sebum, sweat, and biofilm, EPS, etc. Poly-organic stains composes different organic molecules such as polysaccharides, extracellular DNA (exDNA), mannan, starch and proteins. Some biofilm EPS in particular from fungi may comprise polysaccharide constituents such as α-mannan, β-1,6 glucan, and β-1,3 glucan. Some organic matter may be sticky or glueing, which when present on textile, attracts soils and may course redeposition or backstaining of soil resulting in a greying of the textile. Additionally, polymeric substances such as EPS often cause malodor issue as various malodor molecules can be adhered by the polysaccharides, extracellular DNA (exDNA), and proteins in the complex extracellular matrix and be slowly released to cause consumer noticeable malodor issue. There is still a need for cleaning compositions, which effectively prevent, reduce or remove stains. The present invention provides new compositions fulfilling such need.

### SUMMARY OF THE INVENTION

A first aspect of the present invention relates to a cleaning composition comprising a DNase, an alpha-mannan degrading enzyme and at least one cleaning component.

A second aspect of the invention relates to the use of a cleaning composition comprising a DNase, an alpha-mannan degrading enzyme and at least one cleaning component for deep cleaning of an item, wherein the item is a textile or a surface.

A third aspect of the invention relates to a method of cleaning of an item, comprising the steps of:
a) contacting the item with a cleaning composition comprising a DNase, an alpha-mannan degrading enzyme and at least one cleaning component; and
b) optionally rinsing the item, wherein the item is preferably a textile.

### DETAILED DESCRIPTION OF THE INVENTION

Various enzymes are applied in cleaning processes each targeting specific types of stains such as protein, starch and grease stains. Enzymes are now standard ingredients in detergents for laundry and dish wash. The effectiveness of these commercial enzymes provides detergents which removes much of the soiling. However, components of organic matters such as body soils, e.g. dead skin cells, cell debris, sweat, biofilm EPS (extracellular polymeric substance) components and pollution components constitute a challenging type of staining due to the complex nature of such organic stains. None of the commercially available cleaning compositions effectively remove or reduce such complex stains. Polysaccharides, mannan and macromolecules such as DNA are difficult to remove with the traditional cleaning compositions, when all mixed in a poly organic stain. The poly-organic comprising polysaccharides and DNA may also have glue effects when such stains stick to e.g. laundry textile as well as coursing malodor. A poly-organic stain is in the context of the present invention a stain comprising more than one organic component such as stains from body soiling e.g. skin cell debris, sebum, sweat, and biofilm, EPS, etc. which comprises several organic molecules such as polysaccharides, extracellular DNA (exDNA), mannan e.g. α-mannan, starch and proteins.

The compositions of the invention comprise a blend of DNase and alpha-mannan degrading enzyme and effectively reduce or remove organic components, such as mannan and DNA from surfaces such as textiles and hard surfaces e.g. dishes.

The compositions of the invention comprise a blend of DNase and alpha-mannan degrading enzyme and effectively reduce or limit redeposition when applied in e.g. laundry process.

The compositions of the invention comprise a blend of DNase and alpha-mannan degrading enzyme and effectively reduce or limit malodor of e.g. textiles or hard surfaces such as dishes.

The compositions of the invention comprise a blend of DNase and alpha-mannan degrading enzyme and improve whiteness of textile.

A composition of the invention is preferably a cleaning composition, the composition of the invention comprises at least one DNase and at least one alpha-mannan degrading enzyme. Examples of useful DNases and alpha-mannan degrading enzymes are mentioned below in the sections “Polypeptides having DNase activity” and “Polypeptides having alpha-mannan degrading enzyme activity” respectively.

### Polypeptides Having DNase Activity

The term “DNase” means a polypeptide with DNase (deoxyribonuclease) activity that catalyzes the hydrolytic cleavage of phosphodiester linkages in a DNA backbone, thus degrading DNA. Exodeoxyribonuclease cut or cleaves residues at the end of the DNA back bone where endo-deoxyribonucleases cleaves or cut within the DNA backbone. A DNase may cleave only double-stranded DNA or may cleave double stranded and single stranded DNA. The term “DNases” and the expression “a polypeptide with DNase activity” may be used interchangeably throughout the application. For purposes of the present invention, DNase activity is determined according to the procedure described in the Assay I or II.

Preferably the DNase is selected from any of the enzyme classes E.C.3.1, preferably E.C.3.1.21, e.g. such as E.C.3.1.21.X, where X=1, 2, 3, 4, 5, 6, 7, 8 or 9, or e.g. Deoxyribonuclease 1, Deoxyribonuclease IV, Type I site-specific deoxyribonuclease, Type II site-specific deoxyribonuclease, Type III site-specific deoxyribonuclease, CC-preferring endo-deoxyribonuclease, Deoxyribonuclease V, T(4) deoxyribonuclease II, T(4) deoxyribonuclease IV or E.C. 3.1.22.Y where Y=1, 2, 4 or 5, e.g. Deoxyribonuclease II, Aspergillus deoxyribonuclease K(1), Crossover junction endo-deoxyribonuclease, Deoxyribonuclease X.

Preferably, the polypeptide having DNase activity is obtained from a microorganism and the DNase is a microbial enzyme. The DNase is preferably of fungal or bacterial origin.

The DNase may be obtainable from Bacillus e.g. such as a Bacillus licheniformis, Bacillus subtilis, Bacillus horikoshii, Bacillus horneckiae, Bacillus cibi, Bacillus idriensis, Bacillus algicola, Bacillus vietnamensis, Bacillus hwajinpoensis, Bacillus indicus, Bacillus marisflavi, Bacillus luciferensis.

The DNase may also be obtained from any of the following Pyrenochaetopsis sp., Vibrissea flavovirens, Setosphaeria rostrate, Endophragmiella valdina, Corynespora cassiicola, Paraphoma sp., Monilinia fructicola, Curvularia lunata, Penicillium reticulisporum, Penicillium quercetorum, Setophaeosphaeria sp., Alternaria, Alternaria sp., Trichoderma reesei, Chaetomium thermophilum, Scytalidium thermophilum, Metapochonia suchlasporia, Daldinia fissa, Acremonium sp., Acremonium dichromosporum, Sarocladium sp., Metarhizium sp., Isaria tenuipes Scytalidium circinatum, Metarhizium lepidiotae, Thermobispora bispora, Sporormia fimetaria, Pycnidiophora cf. dispera, Clavicipitaceae sp., Westerdykella sp., Humicolopsis cephalosporioides, Neosartorya massa, Roussoella intermedia, Pleosporales, Phaeosphaeria or Didymosphaeria futilis.

In one embodiment the DNases to be used in a composition of the invention preferable belong to the NUC1 group of DNases. The NUC1 group of DNases comprises polypeptides which in addition to having DNase activity, may comprise one or more of the motifs [T/D/S][G/N]PQL (SEQ ID NO: 69), [F/L/Y/I]A[N/R]D[L/I/P/V] (SEQ ID NO: 70), or C[D/N]T[A/R] (SEQ ID NO: 71). One embodiment of the invention relates to a composition comprising an alpha-mannan degrading enzyme and a polypeptide having DNase activity, wherein the polypeptide comprises one or more of the motifs [T/D/S][G/N]PQL (SEQ ID NO: 69), [F/L/Y/I]A[N/R]D[L/I/P/V] (SEQ ID NO: 70) and/or C[D/N]T[A/R] (SEQ ID NO: 71).

The DNases preferably comprise a NUC1_A domain [D/Q][I/V]DH (SEQ ID NO: 72). In addition to comprising any of the domain motifs [T/D/S][G/N]PQL, [F/L/Y/I]A[N/R]D[L/I/P/V] or C[D/N]T[A/R] the polypeptides having DNase activity, to be used in a composition of the invention, may comprise the NUC1_A domain and may share the common motif [D/Q][I/V]DH (SEQ ID NO: 72). One embodiment the invention relates to compositions comprising an alpha-mannan degrading enzyme and polypeptides, which comprises one or more motifs selected from the motifs [T/D/S][G/N]PQL, [F/L/Y/I]A[N/R]D[L/I/P/V], C[D/N]T[A/R] and [D/Q][I/V]DH, wherein the polypeptides have DNase activity.

The DNases to be added to a composition of the invention preferably belong to the group of DNases comprised in the GYS-clade, which are group of DNases on the same branch of a phylogenetic tree having both structural and functional similarities. These DNases which may be defined as NUC1 and/or NUC1_A DNases comprise the conservative motifs [D/M/L][S/T]GYSR[D/N] (SEQ ID NO: 73) or ASXNRSKG (SEQ ID NO: 74) and share similar structural and functional properties. The DNases of the GYS-clade are preferably obtained from Bacillus genus.

One embodiment of the invention relates to a composition comprising an alpha-mannan degrading enzyme and a polypeptide of the GYS clade having DNase activity, optionally wherein the polypeptide comprises one or both of the motifs [D/M/L][S/T]GYSR[D/N] (SEQ ID NO: 73), ASXNRSKG (SEQ ID NO: 74) and wherein the polypeptide is selected from the group of polypeptides:
a) a polypeptide having at least 60%, at least 65%, at least 70%, at least 75% at least 80%, at least 85%, at least 90%, at least 95%, at least 98% or 100% sequence identity to the polypeptide shown in SEQ ID NO: 1,
b) a polypeptide having at least 60%, at least 65%, at least 70%, at least 75% at least 80%, at least 85%, at least 90%, at least 95%, at least 98% or 100% sequence identity to the polypeptide shown in SEQ ID NO: 2,
c) a polypeptide having at least 60%, at least 65%, at least 70%, at least 75% at least 80%, at least 85%, at least 90%, at least 95%, at least 98% or 100% sequence identity to the polypeptide shown in SEQ ID NO: 3,
d) a polypeptide having at least 60%, at least 65%, at least 70%, at least 75% at least 80%, at least 85%, at least 90%, at least 95%, at least 98% or 100% sequence identity to the polypeptide shown in SEQ ID NO: 4,
e) a polypeptide having at least 60%, at least 65%, at least 70%, at least 75% at least 80%, at least 85%, at least 90%, at least 95%, at least 98% or 100% sequence identity to the polypeptide shown in SEQ ID NO: 5,
f) a polypeptide having at least 60%, at least 65%, at least 70%, at least 75% at least 80%, at least 85%, at least 90%, at least 95%, at least 98% or 100% sequence identity to the polypeptide shown in SEQ ID NO: 6,
g) a polypeptide having at least 60%, at least 65%, at least 70%, at least 75% at least 80%, at least 85%, at least 90%, at least 95%, at least 98% or 100% sequence identity to the polypeptide shown in SEQ ID NO: 7,
h) a polypeptide having at least 60%, at least 65%, at least 70%, at least 75% at least 80%, at least 85%, at least 90%, at least 95%, at least 98% or 100% sequence identity to the polypeptide shown in SEQ ID NO: 8,
i) a polypeptide having at least 60%, at least 65%, at least 70%, at least 75% at least 80%, at least 85%, at least 90%, at least 95%, at least 98% or 100% sequence identity to the polypeptide shown in SEQ ID NO: 9,
j) a polypeptide having at least 60%, at least 65%, at least 70%, at least 75% at least 80%, at least 85%, at least 90%, at least 95%, at least 98% or 100% sequence identity to the polypeptide shown in SEQ ID NO: 10,
k) a polypeptide having at least 60%, at least 65%, at least 70%, at least 75% at least 80%, at least 85%, at least 90%, at least 95%, at least 98% or 100% sequence identity to the polypeptide shown in SEQ ID NO: 11,
l) a polypeptide having at least 60%, at least 65%, at least 70%, at least 75% at least 80%, at least 85%, at least 90%, at least 95%, at least 98% or 100% sequence identity to the polypeptide shown in SEQ ID NO: 12,
m) a polypeptide having at least 60%, at least 65%, at least 70%, at least 75% at least 80%, at least 85%, at least 90%, at least 95%, at least 98% or 100% sequence identity to the polypeptide shown in SEQ ID NO: 13,
n) a polypeptide having at least 60%, at least 65%, at least 70%, at least 75% at least 80%, at least 85%, at least 90%, at least 95%, at least 98% or 100% sequence identity to the polypeptide shown in SEQ ID NO: 14,
o) a polypeptide having at least 60%, at least 65%, at least 70%, at least 75% at least 80%, at least 85%, at least 90%, at least 95%, at least 98% or 100% sequence identity to the polypeptide shown in SEQ ID NO: 15,
p) a polypeptide having at least 60%, at least 65%, at least 70%, at least 75% at least 80%, at least 85%, at least 90%, at least 95%, at least 98% or 100% sequence identity to the polypeptide shown in SEQ ID NO: 16,
q) a polypeptide having at least 60%, at least 65%, at least 70%, at least 75% at least 80%, at least 85%, at least 90%, at least 95%, at least 98% or 100% sequence identity to the polypeptide shown in SEQ ID NO: 17,
r) a polypeptide having at least 60%, at least 65%, at least 70%, at least 75% at least 80%, at least 85%, at least 90%, at least 95%, at least 98% or 100% sequence identity to the polypeptide shown in SEQ ID NO: 18,
s) a polypeptide having at least 60%, at least 65%, at least 70%, at least 75% at least 80%, at least 85%, at least 90%, at least 95%, at least 98% or 100% sequence identity to the polypeptide shown in SEQ ID NO: 19,
t) a polypeptide having at least 60%, at least 65%, at least 70%, at least 75% at least 80%, at least 85%, at least 90%, at least 95%, at least 98% or 100% sequence identity to the polypeptide shown in SEQ ID NO: 20,
u) a polypeptide having at least 60%, at least 65%, at least 70%, at least 75% at least 80%, at least 85%, at least 90%, at least 95%, at least 98% or 100% sequence identity to the polypeptide shown in SEQ ID NO: 21,
v) a polypeptide having at least 60%, at least 65%, at least 70%, at least 75% at least 80%, at least 85%, at least 90%, at least 95%, at least 98% or 100% sequence identity to the polypeptide shown in SEQ ID NO: 22,
w) a polypeptide having at least 60%, at least 65%, at least 70%, at least 75% at least 80%, at least 85%, at least 90%, at least 95%, at least 98% or 100% sequence identity to the polypeptide shown in SEQ ID NO: 23,
x) a polypeptide having at least 60%, at least 65%, at least 70%, at least 75% at least 80%, at least 85%, at least 90%, at least 95%, at least 98% or 100% sequence identity to the polypeptide shown in SEQ ID NO: 24, and
y) a polypeptide having at least 60%, at least 65%, at least 70%, at least 75% at least 80%, at least 85%, at least 90%, at least 95%, at least 98% or 100% sequence identity to the polypeptide shown in SEQ ID NO: 25.

Polypeptides having DNase activity and which comprise the GYS-clade motifs have shown particularly good deep cleaning properties e.g. the DNases are particularly effective in removing or reducing components of organic matter, such as DNA, from an item such as a textile or a hard surface. In addition, these DNases are particularly effective in removing or reducing malodor, from an item such as a textile or a hard surface. Further, the GYS-clade DNases are particularly effective in preventing redeposition when laundering an item such as textile.

In one embodiment the DNases to be added in a composition of the invention preferably belong to the group of DNases comprised in the NAWK-clade, which may be defined as NUC1 and NUC1_A DNases, which may further comprise the conservative motifs [V/I]PL[S/A]NAWK (SEQ ID NO: 75) or NPQL (SEQ ID NO: 76).

One embodiment of the invention relates to a composition comprising an alpha-mannan degrading enzyme and a polypeptide of the NAWK-clade having DNase activity, optionally wherein the polypeptide comprises one or both of the motifs [V/I]PL[S/A]NAWK (SEQ ID NO: 75) or NPQL (SEQ ID NO: 76) and wherein the polypeptide is selected from the group of polypeptides:
a) a polypeptide having at least 60%, at least 65%, at least 70%, at least 75% at least 80%, at least 85%, at least 90%, at least 95%, at least 98% or 100% sequence identity to the polypeptide shown in SEQ ID NO: 26,
b) a polypeptide having at least 60%, at least 65%, at least 70%, at least 75% at least 80%, at least 85%, at least 90%, at least 95%, at least 98% or 100% sequence identity to the polypeptide shown in SEQ ID NO: 27,
c) a polypeptide having at least 60%, at least 65%, at least 70%, at least 75% at least 80%, at least 85%, at least 90%, at least 95%, at least 98% or 100% sequence identity to the polypeptide shown in SEQ ID NO: 28,
d) a polypeptide having at least 60%, at least 65%, at least 70%, at least 75% at least 80%, at least 85%, at least 90%, at least 95%, at least 98% or 100% sequence identity to the polypeptide shown in SEQ ID NO: 29,
e) a polypeptide having at least 60%, at least 65%, at least 70%, at least 75% at least 80%, at least 85%, at least 90%, at least 95%, at least 98% or 100% sequence identity to the polypeptide shown in SEQ ID NO: 30,
f) a polypeptide having at least 60%, at least 65%, at least 70%, at least 75% at least 80%, at least 85%, at least 90%, at least 95%, at least 98% or 100% sequence identity to the polypeptide shown in SEQ ID NO: 31,
g) a polypeptide having at least 60%, at least 65%, at least 70%, at least 75% at least 80%, at least 85%, at least 90%, at least 95%, at least 98% or 100% sequence identity to the polypeptide shown in SEQ ID NO: 32,
h) a polypeptide having at least 60%, at least 65%, at least 70%, at least 75% at least 80%, at least 85%, at least 90%, at least 95%, at least 98% or 100% sequence identity to the polypeptide shown in SEQ ID NO: 33,
i) a polypeptide having at least 60%, at least 65%, at least 70%, at least 75% at least 80%, at least 85%, at least 90%, at least 95%, at least 98% or 100% sequence identity to the polypeptide shown in SEQ ID NO: 34,
j) a polypeptide having at least 60%, at least 65%, at least 70%, at least 75% at least 80%, at least 85%, at least 90%, at least 95%, at least 98% or 100% sequence identity to the polypeptide shown in SEQ ID NO: 35,
k) a polypeptide having at least 60%, at least 65%, at least 70%, at least 75% at least 80%, at least 85%, at least 90%, at least 95%, at least 98% or 100% sequence identity to the polypeptide shown in SEQ ID NO: 36,
l) a polypeptide having at least 60%, at least 65%, at least 70%, at least 75% at least 80%, at least 85%, at least 90%, at least 95%, at least 98% or 100% sequence identity to the polypeptide shown in SEQ ID NO: 37, and
m) a polypeptide having at least 60%, at least 65%, at least 70%, at least 75% at least 80%, at least 85%, at least 90%, at least 95%, at least 98% or 100% sequence identity to the polypeptide shown in SEQ ID NO: 38.

Polypeptides having DNase activity and which comprise the NAWK-clade motifs have shown particularly good deep cleaning properties e.g. the DNases are particularly effective in removing or reducing components of organic matter, such as DNA, from an item such as a textile or a hard surface. In addition, these DNases are particularly effective in removing or reducing malodor, from an item such as a textile or a hard surface. Further, the NAWK-clade DNases are particularly effective in preventing redeposition when laundering an item such as textile.

The DNases to be added in a composition of the invention preferably belong to the group of DNases comprised in the KNAW-clade, which may be defined as NUC1 and NUC1_A DNases which may further comprise the conservative motifs P[Q/E]L[W/Y] (SEQ ID NO: 77) or [K/H/E]NAW (SEQ ID NO: 78).

One embodiment of the invention relates to a composition comprising an alpha-mannan degrading enzyme and a polypeptide of the KNAW clade having DNase activity, optionally wherein the polypeptide comprises one or both of the motifs P[Q/E]L[W/Y] (SEQ ID NO: 77) or [K/H/E]NAW (SEQ ID NO: 78), and wherein the polypeptide is selected from the group of polypeptides:
a) a polypeptide having at least 60%, at least 65%, at least 70%, at least 75% at least 80%, at least 85%, at least 90%, at least 95%, at least 98% or 100% sequence identity to the polypeptide shown in SEQ ID NO: 39,
b) a polypeptide having at least 60%, at least 65%, at least 70%, at least 75% at least 80%, at least 85%, at least 90%, at least 95%, at least 98% or 100% sequence identity to the polypeptide shown in SEQ ID NO: 40,
c) a polypeptide having at least 60%, at least 65%, at least 70%, at least 75% at least 80%, at least 85%, at least 90%, at least 95%, at least 98% or 100% sequence identity to the polypeptide shown in SEQ ID NO: 41,
d) a polypeptide having at least 60%, at least 65%, at least 70%, at least 75% at least 80%, at least 85%, at least 90%, at least 95%, at least 98% or 100% sequence identity to the polypeptide shown in SEQ ID NO: 42,
e) a polypeptide having at least 60%, at least 65%, at least 70%, at least 75% at least 80%, at least 85%, at least 90%, at least 95%, at least 98% or 100% sequence identity to the polypeptide shown in SEQ ID NO: 43
f) a polypeptide having at least 60%, at least 65%, at least 70%, at least 75% at least 80%, at least 85%, at least 90%, at least 95%, at least 98% or 100% sequence identity to the polypeptide shown in SEQ ID NO: 44,
g) a polypeptide having at least 60%, at least 65%, at least 70%, at least 75% at least 80%, at least 85%, at least 90%, at least 95%, at least 98% or 100% sequence identity to the polypeptide shown in SEQ ID NO: 45,
h) a polypeptide having at least 60%, at least 65%, at least 70%, at least 75% at least 80%, at least 85%, at least 90%, at least 95%, at least 98% or 100% sequence identity to the polypeptide shown in SEQ ID NO: 46,
i) a polypeptide having at least 60%, at least 65%, at least 70%, at least 75% at least 80%, at least 85%, at least 90%, at least 95%, at least 98% or 100% sequence identity to the polypeptide shown in SEQ ID NO: 47,
j) a polypeptide having at least 60%, at least 65%, at least 70%, at least 75% at least 80%, at least 85%, at least 90%, at least 95%, at least 98% or 100% sequence identity to the polypeptide shown in SEQ ID NO: 48,
k) a polypeptide having at least 60%, at least 65%, at least 70%, at least 75% at least 80%, at least 85%, at least 90%, at least 95%, at least 98% or 100% sequence identity to the polypeptide shown in SEQ ID NO: 49,
l) a polypeptide having at least 60%, at least 65%, at least 70%, at least 75% at least 80%, at least 85%, at least 90%, at least 95%, at least 98% or 100% sequence identity to the polypeptide shown in SEQ ID NO: 50, and
m) a polypeptide having at least 60%, at least 65%, at least 70%, at least 75% at least 80%, at least 85%, at least 90%, at least 95%, at least 98% or 100% sequence identity to the polypeptide shown in SEQ ID NO: 51.

Polypeptides having DNase activity and which comprise the KNAW-clade motifs have shown particularly good deep cleaning properties e.g. the DNases are particularly effective in removing or reducing components of organic matter, such as DNA, from an item such as a textile or a hard surface. In addition, these DNases are particularly effective in removing or reducing malodor, from an item such as a textile or a hard surface. Further, the KNAW-clade DNases are particularly effective in preventing redeposition when laundering an item such as textile.

The DNases of the GYS, NAWK and KNAW-clades are also described in WO2017/060475 (Novozymes A/S).

In some embodiments, the present invention relates compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 1 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 1.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus horikoshii, having a sequence identity to the polypeptide shown in SEQ ID NO: 2 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 2.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 3 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 3.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 4 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 4.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus horikoshii, having a sequence identity to the polypeptide shown in SEQ ID NO: 5 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 5.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus horikoshii, having a sequence identity to the polypeptide shown in SEQ ID NO: 6 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 6.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 7 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 7.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 8 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 8.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 9 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 9.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 10 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 10.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus horneckiae, having a sequence identity to the polypeptide shown in SEQ ID NO: 11 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 11.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 12 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 12.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus cibi, having a sequence identity to the polypeptide shown in SEQ ID NO: 13 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 13.

In some embodiments, the present invention relates to compositions comprising an alpha-mannan degrading enzyme, preferably of the glycosyl hydrolase family GH76, GH92 or GH99, and a polypeptide, preferably obtainable from Bacillus, having a sequence identity to the polypeptide shown in SEQ ID NO: 14 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 14.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus idriensis, having a sequence identity to the polypeptide shown in SEQ ID NO: 15 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 15.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus algicola, having a sequence identity to the polypeptide shown in SEQ ID NO: 16 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 16.

In some embodiments, the present invention relates to compositions comprising an alpha-mannan degrading enzyme, preferably of the glycosyl hydrolase family GH76, GH92 or GH99, and a polypeptide having a sequence identity to the polypeptide shown in SEQ ID NO: 17 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 17.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus vietnamensis, having a sequence identity to the polypeptide shown in SEQ ID NO: 18 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 18.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus hwajinpoensis, having a sequence identity to the polypeptide shown in SEQ ID NO: 19 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 19.

In some embodiments, the present invention relates to compositions comprising an alpha-mannan degrading enzyme, preferably of the glycosyl hydrolase family GH76, GH92 or GH99, and a polypeptide, preferably obtainable from Paenibacillus mucilaginosus, having a sequence identity to the polypeptide shown in SEQ ID NO: 20 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 20.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus indicus, having a sequence identity to the polypeptide shown in SEQ ID NO: 21 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 21.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus marisflavi, having a sequence identity to the polypeptide shown in SEQ ID NO: 22 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 22.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus luciferensis, having a sequence identity to the polypeptide shown in SEQ ID NO: 23 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 23.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus marisflavi, having a sequence identity to the polypeptide shown in SEQ ID NO: 24 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 24.

In some embodiments, the present invention relates to compositions comprising an alpha-mannan degrading enzyme, preferably of the glycosyl hydrolase family GH76, GH92 or GH99, and a polypeptide preferably obtainable from Bacillus, having a sequence identity to the polypeptide shown in SEQ ID NO: 25 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 25.

In some embodiments, the present invention relates to compositions comprising an alpha-mannan degrading enzyme, preferably of the glycosyl hydrolase family GH76, GH92 or GH99, and a polypeptide, preferably obtainable from Pyrenochaetopsis sp., having a sequence identity to the polypeptide shown in SEQ ID NO: 26 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 26.

In some embodiments, the present invention relates to compositions comprising an alpha-mannan degrading enzyme, preferably of the glycosyl hydrolase family GH76, GH92 or GH99, and a polypeptide, preferably obtainable from Vibrissea flavovirens, having a sequence identity to the polypeptide shown in SEQ ID NO: 27 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 27.

In some embodiments, the present invention relates to compositions comprising an alpha-mannan degrading enzyme, preferably of the glycosyl hydrolase family GH76, GH92 or GH99, and a polypeptide, preferably obtainable from Setosphaeria rostrate, having a sequence identity to the polypeptide shown in SEQ ID NO: 28 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 28.

In some embodiments, the present invention relates to compositions comprising an alpha-mannan degrading enzyme, preferably of the glycosyl hydrolase family GH76, GH92 or GH99, and a polypeptide, preferably obtainable from Endophragmiella valdina, having a sequence identity to the polypeptide shown in SEQ ID NO: 29 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 29.

In some embodiments, the present invention relates to compositions comprising an alpha-mannan degrading enzyme, preferably of the glycosyl hydrolase family GH76, GH92 or GH99, and a polypeptide, preferably obtainable from Corynespora cassiicola, having a sequence identity to the polypeptide shown in SEQ ID NO: 30 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 30.

In some embodiments, the present invention relates to compositions comprising an alpha-mannan degrading enzyme, preferably of the glycosyl hydrolase family GH76, GH92 or GH99, and a polypeptide, preferably obtainable from Paraphoma sp. XZ1965, having a sequence identity to the polypeptide shown in SEQ ID NO: 31 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 31.

In some embodiments, the present invention relates to compositions comprising an alpha-mannan degrading enzyme, preferably of the glycosyl hydrolase family GH76, GH92 or GH99, and a polypeptide, preferably obtainable from Monilinia fructicola, having a sequence identity to the polypeptide shown in SEQ ID NO: 32 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 32.

In some embodiments, the present invention relates to compositions comprising an alpha-mannan degrading enzyme, preferably of the glycosyl hydrolase family GH76, GH92 or GH99, and a polypeptide, preferably obtainable from Curvularia lunata, having a sequence identity to the polypeptide shown in SEQ ID NO: 33 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 33.

In some embodiments, the present invention relates to compositions comprising an alpha-mannan degrading enzyme, preferably of the glycosyl hydrolase family GH76, GH92 or GH99, and a polypeptide, preferably obtainable from Penicillium reticulisporum, having a sequence identity to the polypeptide shown in SEQ ID NO: 34 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 34.

In some embodiments, the present invention relates to compositions comprising an alpha-mannan degrading enzyme, preferably of the glycosyl hydrolase family GH76, GH92 or GH99, and a polypeptide, preferably obtainable from Penicillium quercetorum, having a sequence identity to the polypeptide shown in SEQ ID NO: 35 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 35.

In some embodiments, the present invention relates to compositions comprising an alpha-mannan degrading enzyme, preferably of the glycosyl hydrolase family GH76, GH92 or GH99, and a polypeptide, preferably obtainable from Setophaeosphaeria sp., having a sequence identity to the polypeptide shown in SEQ ID NO: 36 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 36.

In some embodiments, the present invention relates to compositions comprising an alpha-mannan degrading enzyme, preferably of the glycosyl hydrolase family GH76, GH92 or GH99, and a polypeptide, preferably obtainable from Alternaria sp., having a sequence identity to the polypeptide shown in SEQ ID NO: 37 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 37.

In some embodiments, the present invention relates to compositions comprising an alpha-mannan degrading enzyme, preferably of the glycosyl hydrolase family GH76, GH92 or GH99, and a polypeptide, preferably obtainable from Alternaria, having a sequence identity to the polypeptide shown in SEQ ID NO: 38 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 38.

In some embodiments, the present invention relates to compositions comprising an alpha-mannan degrading enzyme, preferably of the glycosyl hydrolase family GH76, GH92 or GH99, and a polypeptide, preferably obtainable from Trichoderma reesei, having a sequence identity to the polypeptide shown in SEQ ID NO: 39 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 39.

In some embodiments, the present invention relates to compositions comprising an alpha-mannan degrading enzyme, preferably of the glycosyl hydrolase family GH76, GH92 or GH99, and a polypeptide, preferably obtainable from Chaetomium thermophilum, having a sequence identity to the polypeptide shown in SEQ ID NO: 40 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 40.

In some embodiments, the present invention relates to compositions comprising an alpha-mannan degrading enzyme, preferably of the glycosyl hydrolase family GH76, GH92 or GH99, and a polypeptide, preferably obtainable from Scytalidium thermophilum, having a sequence identity to the polypeptide shown in SEQ ID NO: 41 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 41.

In some embodiments, the present invention relates to compositions comprising an alpha-mannan degrading enzyme, preferably of the glycosyl hydrolase family GH76, GH92 or GH99, and a polypeptide, preferably obtainable from Metapochonia suchlasporia, having a sequence identity to the polypeptide shown in SEQ ID NO: 42 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 42.

In some embodiments, the present invention relates to compositions comprising an alpha-mannan degrading enzyme, preferably of the glycosyl hydrolase family GH76, GH92 or GH99, and a polypeptide, preferably obtainable from Daldinia fissa, having a sequence identity to the polypeptide shown in SEQ ID NO: 43 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 43.

In some embodiments, the present invention relates to compositions comprising an alpha-mannan degrading enzyme, preferably of the glycosyl hydrolase family GH76, GH92 or GH99, and a polypeptide, preferably obtainable from Acremonium sp., having a sequence identity to the polypeptide shown in SEQ ID NO: 44 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 44.

In some embodiments, the present invention relates to compositions comprising an alpha-mannan degrading enzyme, preferably of the glycosyl hydrolase family GH76, GH92 or GH99, and a polypeptide, preferably obtainable from Acremonium dichromosporum, having a sequence identity to the polypeptide shown in SEQ ID NO: 45 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 45.

In some embodiments, the present invention relates to compositions comprising an alpha-mannan degrading enzyme, preferably of the glycosyl hydrolase family GH76, GH92 or GH99, and a polypeptide, preferably obtainable from Sarocladium sp., having a sequence identity to the polypeptide shown in SEQ ID NO: 46 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 46.

In some embodiments, the present invention relates to compositions comprising an alpha-mannan degrading enzyme, preferably of the glycosyl hydrolase family GH76, GH92 or GH99, and a polypeptide, preferably obtainable from Metarhizium sp., having a sequence identity to the polypeptide shown in SEQ ID NO: 47 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 47.

In some embodiments, the present invention relates to compositions comprising an alpha-mannan degrading enzyme, preferably of the glycosyl hydrolase family GH76, GH92 or GH99, and a polypeptide, preferably obtainable from Acremonium sp., having a sequence identity to the polypeptide shown in SEQ ID NO: 48 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 48.

In some embodiments, the present invention relates to compositions comprising an alpha-mannan degrading enzyme, preferably of the glycosyl hydrolase family GH76, GH92 or GH99, and a polypeptide, preferably obtainable from Isaria tenuipes, having a sequence identity to the polypeptide shown in SEQ ID NO: 49 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 49.

In some embodiments, the present invention relates to compositions comprising an alpha-mannan degrading enzyme, preferably of the glycosyl hydrolase family GH76, GH92 or GH99, and a polypeptide, preferably obtainable from Scytalidium circinatum, having a sequence identity to the polypeptide shown in SEQ ID NO: 50 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 50.

In some embodiments, the present invention relates to compositions comprising an alpha-mannan degrading enzyme, preferably of the glycosyl hydrolase family GH76, GH92 or GH99, and a polypeptide, preferably obtainable from Metarhizium lepidiotae, having a sequence identity to the polypeptide shown in SEQ ID NO: 51 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 51.

In some embodiments, the present invention relates to compositions comprising an alpha-mannan degrading enzyme, preferably of the glycosyl hydrolase family GH76, GH92 or GH99, and a polypeptide, preferably obtainable from Thermobispora bispora, having a sequence identity to the polypeptide shown in SEQ ID NO: 52 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 52.

In some embodiments, the present invention relates to compositions comprising an alpha-mannan degrading enzyme, preferably of the glycosyl hydrolase family GH76, GH92 or GH99, and a polypeptide, preferably obtainable from Sporormia fimetaria, having a sequence identity to the polypeptide shown in SEQ ID NO: 53 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 53.

In some embodiments, the present invention relates to compositions comprising an alpha-mannan degrading enzyme, preferably of the glycosyl hydrolase family GH76, GH92 or GH99, and a polypeptide, preferably obtainable from Pycnidiophora cf. dispera, having a sequence identity to the polypeptide shown in SEQ ID NO: 54 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 54.

In some embodiments, the present invention relates to compositions comprising an alpha-mannan degrading enzyme, preferably of the glycosyl hydrolase family GH76, GH92 or GH99, and a polypeptide, having a sequence identity to the polypeptide shown in SEQ ID NO: 55 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 55.

In some embodiments, the present invention relates to compositions comprising an alpha-mannan degrading enzyme, preferably of the glycosyl hydrolase family GH76, GH92 or GH99, and a polypeptide, having a sequence identity to the polypeptide shown in SEQ ID NO: 56 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 56.

In some embodiments, the present invention relates to compositions comprising an alpha-mannan degrading enzyme, preferably of the glycosyl hydrolase family GH76, GH92 or GH99, and a polypeptide, preferably obtainable from Clavicipitaceae, having a sequence identity to the polypeptide shown in SEQ ID NO: 57 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 57.

In some embodiments, the present invention relates to compositions comprising an alpha-mannan degrading enzyme, preferably of the glycosyl hydrolase family GH76, GH92 or GH99, and a polypeptide, preferably obtainable from Westerdykella sp., having a sequence identity to the polypeptide shown in SEQ ID NO: 58 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 58.

In some embodiments, the present invention relates to compositions comprising an alpha-mannan degrading enzyme, preferably of the glycosyl hydrolase family GH76, GH92 or GH99, and a polypeptide, preferably obtainable from Humicolopsis cephalosporioides, having a sequence identity to the polypeptide shown in SEQ ID NO: 59 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 59.

In some embodiments, the present invention relates to compositions comprising an alpha-mannan degrading enzyme, preferably of the glycosyl hydrolase family GH76, GH92 or GH99, and a polypeptide, preferable obtainable from Neosartorya massa, having a sequence identity to the polypeptide shown in SEQ ID NO: 60 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 60.

In some embodiments, the present invention relates to compositions comprising an alpha-mannan degrading enzyme, preferably of the glycosyl hydrolase family GH76, GH92 or GH99, and a polypeptide, preferably obtainable from Roussoella intermedia, having a sequence identity to the polypeptide shown in SEQ ID NO: 61 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 61.

In some embodiments, the present invention relates to compositions comprising an alpha-mannan degrading enzyme, preferably of the glycosyl hydrolase family GH76, GH92 or GH99, and a polypeptide, preferably obtainable from Pleosporales, having a sequence identity to the polypeptide shown in SEQ ID NO: 62 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 62.

In some embodiments, the present invention relates to compositions comprising an alpha-mannan degrading enzyme, preferably of the glycosyl hydrolase family GH76, GH92 or GH99, and a polypeptide, preferably obtainable from Phaeosphaeria, having a sequence identity to the polypeptide shown in SEQ ID NO: 63 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 63.

In some embodiments, the present invention relates to compositions comprising an alpha-mannan degrading enzyme, preferably of the glycosyl hydrolase family GH76, GH92 or GH99, and a polypeptide, preferably obtainable from Didymosphaeria futilis, having a sequence identity to the polypeptide shown in SEQ ID NO: 64 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 64.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus licheniformis, having a sequence identity to the polypeptide shown in SEQ ID NO: 65 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 65.

In some embodiments, the present invention relates to compositions comprising an alpha-mannan degrading enzyme, preferably of the glycosyl hydrolase family GH76, GH92 or GH99, and a polypeptide, preferably obtainable from Bacillus e.g. obtainable from Bacillus subtilis, having a sequence identity to the polypeptide shown in SEQ ID NO: 66 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 66.

In some embodiments, the present invention relates to compositions comprising an alpha-mannan degrading enzyme, preferably of the glycosyl hydrolase family GH76, GH92 or GH99, and a polypeptide, preferably obtainable from Aspergillus e.g. obtainable from Aspergillus oryzae, having a sequence identity to the polypeptide shown in SEQ ID NO: 67 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 67.

In some embodiments, the present invention relates to compositions comprising an alpha-mannan degrading enzyme, preferably of the glycosyl hydrolase family GH76, GH92 or GH99, and a polypeptide, preferably obtainable from Trichoderma e.g. obtainable from Trichoderma harzianum, having a sequence identity to the polypeptide shown in SEQ ID NO: 68 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity. In one aspect, the polypeptides differ by up to 10 amino acids, e.g., 1, 2, 3, 4, 5, 6, 7, 8, 9, or 10, from the mature polypeptide shown in SEQ ID NO: 68.

### Polypeptides Having Alpha-Mannan Degrading Enzyme Activity

The term “alpha-mannan degrading enzyme” or “polypeptide having alpha-mannan degrading activity” or alpha-mannanases means an enzyme having hydrolase activity on alpha-mannan. For purposes of the present invention, alpha-mannanase activity is determined according to the procedure described in the Assay Ill. Relevant are enzymes having alpha-mannanase and/or alpha-mannosidase activity. In particular, the polypeptide having alpha-mannan degrading activity includes glycoside hydrolase domains GH76, GH92, or GH99, as defined in CAZY (available at cazy.org, and as described in Lombard V, et al. 2014, Nucleic Acids Res 42:D490-D495). These can include enzyme activities such as alpha-1,6-mannanase (EC 3.2.1.101), alpha-1,2-mannase; mannosyl-oligosaccharide alpha-1,2-mannosidase (EC 3.2.1.113); mannosyl-oligosaccharide alpha-1,3-mannosidase (EC 3.2.1.-); mannosyl-oligosaccharide alpha-1,6-mannosidase (EC 3.2.1.-); alpha-mannosidase (EC 3.2.1.24); alpha-1,2-mannosidase (EC 3.2.1.-); alpha-1,3-mannosidase (EC 3.2.1.-); alpha-1,4-mannosidase (EC 3.2.1.-); mannosyl-1-phosphodiester alpha-1,P-mannosidase (EC 3.2.1.-); glycoprotein endo-alpha-1,2-mannosidase (EC 3.2.1.130); and/or mannan endo-1,2-alpha-mannanase (3.2.1.-) activities. According to the online carbohydrate-active enzyme (“CAZy”) database (available at cazy.org), alpha-mannan degrading enzymes have been found in glycoside hydrolase families including 76, 92, and 99. The present invention provides compositions comprising a polypeptide having DNase activity and polypeptides having alpha-mannanase activity. Polypeptides having alpha-mannan degrading activity or alpha-mannan degrading enzymes are enzymes having hydrolase activity on alpha-mannan. In particular, the polypeptide having alpha-mannan degrading activity includes enzymes from glycoside hydrolase domains GH76, GH92, and GH99, which are enzymes having alpha-mannanase and/or alpha-mannosidase activity. In one embodiment, the polypeptide belongs to GH family 76. In one embodiment, the polypeptide belongs to GH family 92. In one embodiment, the polypeptide belongs to GH family 99.

Also contemplated are compositions comprising DNase with blends of polypeptides having alpha-mannan degrading activity, including, for example, combinations of polypeptides having two or more different GH classifications according to the CAZY naming system.

In an embodiment are provided compositions comprising a DNase and any of the blends selected from the group consisting of:
i. a polypeptide belonging to GH family 76 and a polypeptide belonging to GH family 92;
ii. a polypeptide belonging to GH family 76 and a polypeptide belonging to GH family 99; and
iii. a polypeptide belonging to GH family 92 and a polypeptide belonging to GH family 99.

Also contemplated are blends of three or more different GH classifications according to the CAZY naming system, including blends comprising a polypeptide belonging to GH family 76, a polypeptide belonging to GH family 92, and a polypeptide belonging to GH family 99.

In some embodiments, the present invention relates compositions e.g. cleaning compositions, comprising a DNase and a polypeptide selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 79 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 80 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 81 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 82 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 83 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 84 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 85 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 86 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 87 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 88 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 89 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 90 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 91 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 92 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 93 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 94 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 95 or a fragment thereof having alpha-mannan degrading activity;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 96 or a fragment thereof having alpha-mannan degrading activity, and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 97 or a fragment thereof having alpha-mannan degrading activity.

### Cleaning Compositions

The invention relates to cleaning compositions comprising a DNase and an alpha-mannan degrading enzyme in combination with one or more additional cleaning composition components. One embodiment of the invention relates to a cleaning composition comprising a DNase, an alpha-mannan degrading enzyme and at least one cleaning component. The alpha-mannan degrading enzyme may be any of the alpha-mannan degrading enzymes mentioned under the heading “Polypeptides having alpha-mannan degrading enzyme activity”. Preferably the alpha-mannan degrading enzyme is of (belongs to) the glycosyl hydrolase family GH76, GH92 or GH99.

One embodiment relates to a cleaning composition comprising a DNase, an alpha-mannan degrading enzyme and at least one cleaning component, wherein the alpha-mannan degrading enzyme is of the GH family GH76, GH92 or GH99.

One embodiment relates to a cleaning composition comprising a DNase, an alpha-mannan degrading enzyme and at least one cleaning component, wherein the alpha-mannan degrading enzyme is a GH76 glycosyl hydrolase. One embodiment relates to a cleaning composition comprising a DNase, an alpha-mannan degrading enzyme and at least one cleaning component, wherein the alpha-mannan degrading enzyme is a GH92 glycosyl hydrolase. One embodiment relates to a cleaning composition comprising a DNase, an alpha-mannan degrading enzyme and at least one cleaning component, wherein the alpha-mannan degrading enzyme is a GH99 glycosyl hydrolase.

The most relevant alpha-mannan degrading enzymes for cleaning are those belonging to the glycosyl hydrolase families GH76, GH92 or GH99, such alpha-mannan degrading enzymes have shown to be active in detergents and to effectively remove mannan. Further, the alpha-mannan degrading enzymes acts synergistically with the DNase in poly-organic stains e.g. in reduction, and removal of biofilm or components hereof e.g. DNA and/or mannan. Biofilm EPS is a complex structure comprising e.g. polysaccharides and DNA, the target substrate e.g. the DNA may be embedded in the biofilm structure and it's believed that when the DNases and alpha-mannan degrading enzymes are acting together, the DNA and mannan components are more effectively removed. It is thus advantageous to formulate DNases with alpha-mannan degrading enzymes in cleaning compositions e.g. for laundry. One aspect of the invention relates to a method of formulating a cleaning composition comprising adding a DNase, an alpha-mannan degrading enzyme and at least one cleaning component. The invention further relates to a kit intended for deep cleaning, wherein the kit comprises a solution of an enzyme mixture comprising a DNase and an alpha-mannan degrading enzyme.

The most relevant mannan degrading enzymes used in the cleaning industry today are beta mannanases e.g. of the GH5 or GH26 glycosyl hydrolase families. In contrast, the enzymes of the invention are alpha-mannan degrading enzymes, which have shown to be useful together with the DNases. This is surprising as glycosyl hydrolases degrading alpha mannan are not know for use in cleaning compositions for e.g. laundry and dish wash. Alpha-mannan degrading enzymes suitable for combining with the DNases in the cleaning composition of the invention are preferably glycosyl hydrolases of the GH76, GH92 or GH99 families, which are suitable for cleaning and which has high stain removal capacity under cleaning conditions e.g. in the presence of surfactants, builders or other cleaning components.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequences shown in SEQ ID NOs: 79, 80, 81, 82, 83, 84, 85, 86, 87, 88, 89, 90, 91, 92, 93, 94, 95, 96 or 97.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 79. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 80. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 81. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 82. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 83. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 84. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 85. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 86. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 87. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 88. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 89. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 90. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 91. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 92. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 93. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 94. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 95. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 96. One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 97.

As mentioned the alpha-mannan degrading enzyme should be compatible with cleaning components and likewise, the DNases to be formulated together with the alpha-mannan degrading enzyme or to be used together with the alpha-mannan degrading enzyme should also be compatible with cleaning components. DNases as well as alpha mannanases are at present not standard ingredients in cleaning compositions. However, the applicant has identified DNases suitable for use in cleaning compositions e.g. in WO2017/060475, WO2014/087011, WO2015/155350 and WO2015/155351. Enzymes, such as DNases should not only be compatible with the cleaning components the DNases should also be compatible with other enzymes which may be present in a typical cleaning composition. The inventors have found that the alpha mannan degrading enzymes are compatible with the DNases and preferably are even acting synergistically to remove or reduce complex organic stains (poly-organic stains) or components hereof e.g. extracellular polymeric substances, biofilm, body soils such as skin debris and pollution particles. Particularly useful DNases may be those of microbial origin. One embodiment of the invention relates to a cleaning composition comprising a DNase, an alpha-mannan degrading enzyme, preferably of the glycosyl hydrolase family GH76, GH92 or GH99, and at least one cleaning component, wherein the DNase is microbial, preferably obtained from bacteria or fungi. In one embodiment, the cleaning composition comprise a DNase from bacteria. One embodiment of the invention relates to a cleaning composition comprising a DNase, an alpha-mannan degrading enzyme, preferably of the glycosyl hydrolase family GH76, GH92 or GH99, and at least one cleaning component, wherein the DNase is obtained from Bacillus, preferably Bacillus cibi, Bacillus horikoshii, Bacillus licheniformis, Bacillus subtilis, Bacillus horneckiae, Bacillus idriensis, Bacillus algicola, Bacillus vietnamensis, Bacillus hwajinpoensis, Bacillus indicus, Bacillus marisflavi or Bacillus luciferensis. One embodiment of the invention relates to a cleaning composition comprising a DNase, an alpha-mannan degrading enzyme, preferably of the glycosyl hydrolase family GH76, GH92 or GH99, and at least one cleaning component, wherein the DNase is obtained from Aspergillus, preferably Aspergillus oryzae.

As mentioned above the DNases to be used in a composition of the invention preferable belong to the NUC1 group of DNases. The NUC1 group of DNases may comprise one or more of the motifs [T/D/S][G/N]PQL (SEQ ID NO: 69), [F/L/Y/I]A[N/R]D[L/I/P/V] (SEQ ID NO: 70), or C[D/N]T[A/R] (SEQ ID NO: 71). One embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises one or more of the motifs [T/D/S][G/N]PQL, [F/L/Y/I]A[N/R]D[L/I/P/V] or C[D/N]T[A/R]. The DNases preferably additionally comprise a NUC1_A domain [D/Q][I/V]DH (SEQ ID NO: 72).

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises one or more motifs selected from the motifs [T/D/S][G/N]PQL, [F/L/Y/I]A[N/R]D[L/I/P/V], C[D/N]T[A/R] and [D/Q][I/V]DH.

One preferred embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises two or more motifs selected from the motifs [T/D/S][G/N]PQL, [F/L/Y/I]A[N/R]D[L/I/P/V], C[D/N]T[A/R] and [D/Q][IV]DH.

One preferred embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises three or more motifs selected from the motifs [T/D/S][G/N]PQL, [F/L/Y/I]A[N/R]D[L/I/P/V], C[D/N]T[A/R] and [D/Q][IV]DH.

One preferred embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises all four motifs [T/D/S][G/N]PQL, [F/L/Y/I]A[N/R]D[L/I/P/V], C[D/N]T[A/R] and [D/Q][I/V]DH.

The DNases to be added to a composition of the invention preferably belong to the group of DNases comprised in the GYS-clade, which may be defined as NUC1 and NUC1_A DNases further comprising the conservative motifs [D/M/L][S/T]GYSR[D/N] (SEQ ID NO: 73) or ASXNRSKG (SEQ ID NO: 74) and which share similar structural and functional properties. The DNases of the GYS-clade are preferably obtained from Bacillus genus.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase comprises one or both of the motif(s) [D/M/L][S/T]GYSR[D/N] (SEQ ID NO: 73) or ASXNRSKG (SEQ ID NO: 74). In a particularly preferred embodiment the Bacillus DNase comprises one or both of the motif(s) [D/M/L][S/T]GYSR[D/N] (SEQ ID NO: 73) or ASXNRSKG (SEQ ID NO: 74). In another particularly preferred embodiment the DNase comprises one or both of the motif(s) [D/M/L][S/T]GYSR[D/N] (SEQ ID NO: 73) or ASXNRSKG (SEQ ID NO: 74) and is obtained from Bacillus cibi. In yet another preferred embodiment the DNase comprises the amino acid sequence shown in SEQ ID NO: 13 or DNases closely related hereto.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 13. Other preferred DNases include those comprising the amino acid sequence shown in SEQ ID NO: 65 and 66.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.

The DNase may also preferably be fungal. Particularly preferred are DNases obtained from Aspergillus in particular, Aspergillus oryzae.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.

Other particularly preferred are DNases obtained from Trichoderma in particular, Trichoderma harzianum.

One embodiment of the invention relates to a cleaning composition comprising a DNase, an alpha-mannan degrading enzyme, preferably of the glycosyl hydrolase family GH76, GH92 or GH99, and at least one cleaning component, wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

One embodiment relates to a cleaning composition comprising a Bacillus DNase, an alpha-mannan degrading enzyme and at least one cleaning component, wherein the alpha-mannan degrading enzyme is a GH76 mannanase and wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 89, 90, 91, 92, 93, 94, 95, 96 and 97.

One embodiment relates to a cleaning composition comprising a Bacillus DNase, an alpha-mannan degrading enzyme and at least one cleaning component, wherein the alpha-mannan degrading enzyme is a GH92 mannanase and wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 87.

One embodiment relates to a cleaning composition comprising a Bacillus DNase, an alpha-mannan degrading enzyme and at least one cleaning component, wherein the alpha-mannan degrading enzyme is a GH99 mannanase and wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 88.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 13. One embodiment relates to a cleaning composition comprising a DNase, an alpha-mannan degrading enzyme and at least one cleaning component, wherein the DNase comprises or consists of the polypeptide comprising the amino acid sequence shown in SEQ ID NO: 13 and wherein the alpha-mannan degrading enzyme comprises or consists of the polypeptide comprising the amino acid sequence shown in SEQ ID NO: 79, 80, 81, 82, 83, 84, 85, 86, 87, 88, 89, 90, 91, 92, 93, 94, 95, 96 or 97.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 65. One embodiment relates to a cleaning composition comprising a DNase, an alpha-mannan degrading enzyme and at least one cleaning component, wherein the DNase comprises or consists of the polypeptide comprising the amino acid sequence shown in SEQ ID NO: 65 and wherein the alpha-mannan degrading enzyme comprises or consists of the polypeptide comprising the amino acid sequence shown in SEQ ID NO: 79, 80, 81, 82, 83, 84, 85, 86, 87, 88, 89, 90, 91, 92, 93, 94, 95, 96 or 97.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 66. One embodiment relates to a cleaning composition comprising a DNase, an alpha-mannan degrading enzyme and at least one cleaning component, wherein the DNase comprises or consists of the polypeptide comprising the amino acid sequence shown in SEQ ID NO: 66 and wherein the alpha-mannan degrading enzyme comprises or consists of the polypeptide comprising the amino acid sequence shown in SEQ ID NOs: 79, 80, 81, 82, 83, 84, 85, 86, 87, 88, 89, 90, 91, 92, 93, 94, 95, 96 or 97.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 67. One embodiment relates to a cleaning composition comprising a DNase, an alpha-mannan degrading enzyme and at least one cleaning component, wherein the DNase comprises or consists of the polypeptide comprising the amino acid sequence shown in SEQ ID NO: 67 and wherein the alpha-mannan degrading enzyme comprises or consists of the polypeptide comprising the amino acid sequence shown in SEQ ID NOs: 79, 80, 81, 82, 83, 84, 85, 86, 87, 88, 89, 90, 91, 92, 93, 94, 95, 96 or 97.

One embodiment relates to a cleaning composition comprising a DNase, an alpha-mannan degrading enzyme and at least one cleaning component, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase has at least 60%, at least 65%, at least 70%, at least 75%, at least 80%, at least 85%, at least 90%, at least 95%, at least 98% or 100% sequence identity to the amino acid sequence shown in SEQ ID NO: 68. One embodiment relates to a cleaning composition comprising a DNase, an alpha-mannan degrading enzyme and at least one cleaning component, wherein the DNase comprises or consists of the polypeptide comprising the amino acid sequence shown in SEQ ID NO: 68 and wherein the alpha-mannan degrading enzyme comprises or consists of the polypeptide comprising the amino acid sequence shown in SEQ ID NOs: 79, 80, 81, 82, 83, 84, 85, 86, 87, 88, 89, 90, 91, 92, 93, 94, 95, 96 or 97.

One embodiment of the invention relates to a composition, preferably a cleaning composition, comprising an alpha-mannan degrading enzyme, a polypeptide having DNase activity, wherein the polypeptide belongs to the GYS-clade and comprise one or both of the motifs [D/M/L][S/T]GYSR[D/N] (SEQ ID NO: 73), ASXNRSKG (SEQ ID NO: 74) and wherein the alpha-mannan degrading enzyme belongs to the GH76 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 89, 90, 91, 92, 93, 94, 95, 96 or 97, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the GYS-clade and comprise one or both of the motifs [D/M/L][S/T]GYSR[D/N] (SEQ ID NO: 73), ASXNRSKG (SEQ ID NO: 74) and wherein the alpha-mannan degrading enzyme belongs to the GH92 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 87, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the GYS-clade and comprise one or both of the motifs [D/M/L][S/T]GYSR[D/N] (SEQ ID NO: 73), ASXNRSKG (SEQ ID NO: 74), and wherein the alpha-mannan degrading enzyme belongs to the GH99 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 88, and wherein the composition comprises at least one cleaning component.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 79 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 80 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NO: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 81 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NO: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 82 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 83 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 84 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 85 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 86 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 87 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 88 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 89 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 90 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 91 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 92 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 93 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 94 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 95 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 96 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 1, 2, 3, 4, 5, 6, 7, 8, 9, 10, 11, 12, 13, 14, 15, 16, 17, 18, 19, 20, 21, 22, 23, 24 or 25 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 97 or a polypeptide having at least 60%, at least 70%, at least 80%, at least 90%, at least 95%, at least 98% or 100% sequence identity hereto.

One embodiment of the invention relates to a composition, preferably a cleaning composition, comprising an alpha-mannan degrading enzyme, a polypeptide having DNase activity, wherein the polypeptide belongs to the NAWK-clade and comprise one or both of the motifs [V/I]PL[S/A]NAWK (SEQ ID NO: 75) or NPQL (SEQ ID NO: 76), wherein the alpha-mannan degrading enzyme belongs to the GH76 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 89, 90, 91, 92, 93, 94, 95, 96 or 97, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the NAWK-clade and comprise one or both of the motifs [V/I]PL[S/A]NAWK (SEQ ID NO: 75) or NPQL (SEQ ID NO: 76), wherein the alpha-mannan degrading enzyme belongs to the GH92 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 87, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the NAWK-clade and comprise one or both of the motifs [V/I]PL[S/A]NAWK (SEQ ID NO: 75) or NPQL (SEQ ID NO: 76), wherein the alpha-mannan degrading enzyme belongs to the GH99 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 88, and wherein the composition comprises at least one cleaning component.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NOs: 79 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 80 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 81 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 82 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 83 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 84 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 85 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 86 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 87 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 88 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 89 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 90 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 91 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 92 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 93 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 94 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 95 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 96 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 26, 27, 28, 29, 30, 31, 32, 33, 34, 35, 36, 37 or 38 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 97 or a polypeptide having at least 60%, at least 70%, at least 80%, at least 90%, at least 95%, at least 98% or 100% sequence identity hereto.

One embodiment of the invention relates to a composition, preferably a cleaning composition, comprising an alpha-mannan degrading enzyme, a polypeptide having DNase activity, wherein the polypeptide belongs to the KNAW-clade and comprise one or both of the motifs P[Q/E]L[W/Y] (SEQ ID NO: 77) or [K/H/E]NAW (SEQ ID NO: 78), wherein the alpha-mannan degrading enzyme belongs to the GH76 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 89, 90, 91, 92, 93, 94, 95, 96 or 97, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the KNAW-clade and comprise one or both of the motifs P[Q/E]L[W/Y] (SEQ ID NO: 77) or [K/H/E]NAW (SEQ ID NO: 78), wherein the alpha-mannan degrading enzyme belongs to the GH92 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 87, and wherein the composition comprises at least one cleaning component.

One embodiment of the invention relates to a composition, preferably a cleaning composition, comprising an alpha-mannan degrading enzyme, a polypeptide having DNase activity, wherein the polypeptide belongs to the KNAW-clade and comprise one or both of the motifs P[Q/E]L[W/Y] (SEQ ID NO: 77) or [K/H/E]NAW (SEQ ID NO: 78), wherein the alpha-mannan degrading enzyme belongs to the GH99 glycosyl hydrolase family and preferably wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NO: 88, and wherein the composition comprises at least one cleaning component.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 79 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 80 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 81 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 82 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 83 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 84 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 85 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 86 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 87 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 88 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 89 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 90 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 91 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 92 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 93 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 94 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 95 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 96 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 39, 40, 41, 42, 43, 44, 45, 46, 47, 48, 49, 50 or 51 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 97 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 79 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 80 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 81 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 82 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 83 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 84 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 85 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 86 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 87 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 88 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 89 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 90 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 91 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 92 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 93 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 94 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 95 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 96 or a polypeptide having at least 60%, at least 70%, at least 80%, at least 90%, at least 95%, at least 98% or 100% sequence identity hereto.

One embodiment relates to a cleaning composition comprising a DNase, an alpha-mannan degrading enzyme and at least one cleaning component, wherein the DNase comprises or consists of a polypeptide selected from the group of polypeptides comprising the amino acid sequence shown in SEQ ID NOs: 52, 53, 54, 55, 56, 57, 58, 59, 60, 61, 62, 63, 64, 65, 66, 67 or 68 or a polypeptide having at least 60%, at least 70%, at least 80%, at least 90%, at least 95%, at least 98% or 100% sequence identity hereto and wherein the alpha-mannan degrading enzyme comprises or consists of the polypeptide comprising the amino acid sequence shown in SEQ ID NO: 97 or a polypeptide having at least 60%, at least 70%, at least 80%, at least 90%, at least 95%, at least 98% or 100% sequence identity hereto.

The alpha-mannan degrading enzyme and DNase may be included in the cleaning composition of the present invention at a level of from 0.01 to 1000 ppm, from 1 ppm to 1000 ppm, from 10 ppm to 1000 ppm, from 50 ppm to 1000 ppm, from 100 ppm to 1000 ppm, from 150 ppm to 1000 ppm, from 200 ppm to 1000 ppm, from 250 ppm to 1000 ppm, from 250 ppm to 750 ppm, from 250 ppm to 500 ppm.

The DNases above may be combined with alpha-mannan degrading enzymes to form a blend to be added to the wash liquor solution according to the invention. The concentration of the DNase in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm. The concentration of the alpha-mannan degrading enzyme in the wash liquor solution is typically in the range of wash liquor from 0.00001 ppm to 10 ppm, from 0.00002 ppm to 10 ppm, from 0.0001 ppm to 10 ppm, from 0.0002 ppm to 10 ppm, from 0.001 ppm to 10 ppm, from 0.002 ppm to 10 ppm, from 0.01 ppm to 10 ppm, from 0.02 ppm to 10 ppm, 0.1 ppm to 10 ppm, from 0.2 ppm to 10 ppm, from 0.5 ppm to 5 ppm.
The DNases may be combined with any of the alpha-mannan degrading enzymes below to form a blend to be added to a composition according to the invention.

One embodiment relates to a cleaning composition comprising a DNase, an alpha-mannan degrading enzyme and at least one cleaning component, wherein the amount of DNase in the composition is from 0.01 to 1000 ppm and the amount of alpha-mannan degrading enzyme is from 0.01 to 1000 ppm.

In addition to the alpha-mannan degrading enzyme and DNase the cleaning composition further comprises one or more cleaning component. One embodiment relates to a cleaning composition comprising a DNase, an alpha-mannan degrading enzyme and at least one cleaning component, wherein the cleaning component is selected from surfactants, preferably anionic and/or nonionic, builders and bleach components.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 0.1% to about 15% or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

### Builders and Co-Builders

The cleaning composition may contain about 0-65% by weight, such as about 5% to about 50%, such as about 0.5% to about 20% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The cleaning composition may contain 0-30% by weight, such as about 1% to about 20%, such as about 0.01% to about 10% of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide—urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-KN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

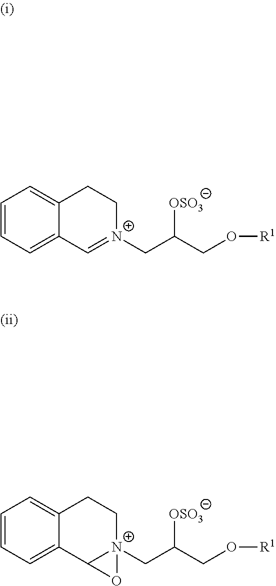

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.
Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

### Metal Care Agents

Metal care agents may prevent or reduce the tarnishing, corrosion or oxidation of metals, including aluminium, stainless steel and non-ferrous metals, such as silver and copper. Suitable examples include one or more of the following:
(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.
(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate;
(c) silicates, including sodium or potassium silicate, sodium disilicate, sodium metasilicate, crystalline phyllosilicate and mixtures thereof.
Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Hydrotropes

The cleaning composition may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The cleaning composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Fabric Hueing Agents

The cleaning compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Enzymes

The cleaning composition may comprise one or more additional enzymes such as one or more lipase, cutinase, an amylase, carbohydrase, cellulase, pectinase, mannanase, arabinase, galactanase, xylanase, oxidase, e.g., a laccase, and/or peroxidase. In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259. Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544. Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147). Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades). Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases include alpha-amylases and/or a glucoamylases and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444. Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184. Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264. Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes A/S), and Rapidase™, Purast™/Effectenz™, Powerase, Preferenz S1000, Preferenz S100 and Preferenz S110 (from Genencor International Inc./DuPont).

### Peroxidases/Oxidases

A peroxidase may be an enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

### Proteases

Suitable proteases include those of bacterial, fungal, plant, viral or animal origin e.g. vegetable or microbial origin. Microbial origin is preferred. Chemically modified or protein engineered mutants are included. The protease may be an alkaline protease, such as a serine protease. A serine protease may for example be of the S1 family, such as trypsin, or the S8 family such as subtilisin. A metalloprotease protease may for example be a thermolysin from e.g. family M4 or other metalloprotease such as those from M5, M7 or M8 families. The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family. Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO09/021867, and Subtilisin lentus, Subtilisin Novo, subtilisin Carlsberg, Bacillus licheniformis, subtilisin BPN′, subtilisin 309, subtilisin 147 and subtilisin 168 and e.g. protease PD138 described in (WO93/18140). Other useful proteases may be those described in WO01/016285 and WO02/016547. Examples of trypsin-like proteases are trypsin (e.g. of porcine or bovine origin) and the Fusarium protease described in WO94/25583 and WO05/040372, and the chymotrypsin proteases derived from Cellumonas described in WO05/052161 and WO05/052146. Further preferred protease is the alkaline protease from Bacillus lentus DSM 5483, as described for example in WO95/23221, and variants thereof which are described in WO92/21760, WO95/23221, EP1921147 and EP1921148. Examples of metalloproteases are the neutral metalloprotease as described in WO07/044993 (Proctor & Gamble/Genencor Int.) such as those derived from Bacillus amyloliquefaciens.

Examples of useful proteases are the variants described in: WO89/06279 WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269, wherein the positions correspond to the positions of the Bacillus lentus protease shown in SEQ ID NO: 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease shown in SEQ ID NO: 1 of WO2016/001449, the Bacillus amylolichenifaciens protease (BPN′) shown in SEQ ID NO: 2 of WO2016/001449. The protease variants preferably have at least 80% sequence identity to SEQ ID NO: 1 or SEQ ID NO: 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™ Purafect Prime®, Preferenz P110™, Effectenz P1000™ Purafect™, Effectenz P1050™ Purafect Ox®™, Effectenz P2000™ Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Dispersants

The cleaning compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The cleaning compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The cleaning compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The cleaning compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The cleaning composition of the present invention may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations. In a specific aspect, the present invention provides a detergent additive comprising one or more enzymes as described herein. The cleaning composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Granular Cleaning Formulations

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The DNase and the alpha-mannan degrading enzyme may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. The multi-enzyme co-granule may comprise an enzyme blend of the invention (alpha-mannanase and DNase) and one or more enzymes selected from the group consisting of alpha-mannan degrading enzymes, lipases, cellulases, xyloglucanases, perhydrolases, peroxidases, lipoxygenases, laccases, hemicellulases, alpha-mannan degrading enzymes, cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof. WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

An embodiment of the invention relates to an enzyme granule/particle comprising the DNase and alpha-mannan degrading enzyme. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm. The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a one embodiment, the thickness of the coating is below 100 μm. In another embodiment, the thickness of the coating is below 60 μm. In an even more particular embodiment the total thickness of the coating is below 40 μm. The coating should encapsulate the core unit by forming a substantially continuous layer. A substantially continuous layer is to be understood as a coating having few or no holes, so that the core unit it is encapsulating/enclosing has few or none uncoated areas. The layer or coating should be homogeneous in thickness. The coating can further contain other materials as known in the art, e.g., fillers, antisticking agents, pigments, dyes, plasticizers and/or binders, such as titanium dioxide, kaolin, calcium carbonate or talc. A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble and may have a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{2° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄-7H₂O), zinc sulfate heptahydrate (ZnSO₄-7H₂O), sodium phosphate dibasic heptahydrate (Na₂HPO₄-7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

One embodiment of the present invention provides a granule, which comprises:

(a) a core comprising a DNase and an alpha-mannan degrading enzyme, preferably selected from GH76, GH92 and GH99, and

(b) optionally a coating consisting of one or more layer(s) surrounding the core.

One embodiment of the invention relates to a granule, which comprises:
(a) a core comprising a DNase and an alpha-mannan degrading enzyme wherein the alpha-mannan degrading enzyme has at least 60%, at least 65%, at least 70%, at least 75%, at least 80%, at least 85%, at least 90%, at least 95%, at least 98% or 100% sequence identity to an amino acid sequence shown in SEQ ID NOs: and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13, and
(b) optionally a coating consisting of one or more layer(s) surrounding the core.

One embodiment of the invention relates to a granule, which comprises:
(a) a core comprising a DNase and an alpha-mannan degrading enzyme wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65, and
(b) optionally a coating consisting of one or more layer(s) surrounding the core.
   One embodiment of the invention relates to a granule, which comprises:
(a) a core comprising a DNase and an alpha-mannan degrading enzyme wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66, and
(b) optionally a coating consisting of one or more layer(s) surrounding the core.
   One embodiment of the invention relates to a granule, which comprises:
(a) a core comprising a DNase and an alpha-mannan degrading enzyme wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67, and
(b) optionally a coating consisting of one or more layer(s) surrounding the core.
   One embodiment of the invention relates to a granule, which comprises:
(a) a core comprising a DNase and an alpha-mannan degrading enzyme wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68, and
(b) optionally a coating consisting of one or more layer(s) surrounding the core.

### Uses

The present invention is also directed to methods for using the compositions thereof. Laundry/textile/fabric (House hold laundry washing, Industrial laundry washing). Hard surface cleaning (ADW, car wash, Industrial surface). The cleaning e.g. detergent composition of the present invention may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations.

The compositions of the invention comprise a blend of DNase and alpha-mannan degrading enzyme and effectively reduce or remove organic components, such as polysaccharides and DNA from surfaces such as textiles and hard surfaces e.g. dishes.

One embodiment of the invention relates to the use of a composition comprising a DNase and alpha-mannan degrading enzyme for reduction of redeposition. One embodiment of the invention relates to the use of a cleaning composition comprising a DNase and alpha-mannan degrading enzyme for reduction of redeposition

One embodiment of the invention relates to the use of a cleaning composition comprising a DNase and alpha-mannan degrading enzyme for reduction of redeposition when the cleaning composition is applied in e.g. laundry process. One embodiment of the invention relates to the use of a cleaning composition comprising a DNase and alpha-mannan degrading enzyme for reduction of redeposition on an item e.g. textile. In one embodiment, the composition is an anti-redeposition composition.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus, preferably Bacillus cibi, Bacillus horikoshii, Bacillus licheniformis, Bacillus subtilis, Bacillus horneckiae, Bacillus idriensis, Bacillus algicola, Bacillus vietnamensis, Bacillus hwajinpoensis, Bacillus indicus, Bacillus marisflavi or Bacillus luciferensis.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 and 97 and wherein the DNase is obtained from Bacillus and comprises one or both of the motif(s) [D/M/L][S/T]GYSR[D/N] (SEQ ID NO: 73) or ASXNRSKG (SEQ ID NO: 74).

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.

One embodiment of the invention relates to the use of a cleaning composition comprising a DNase and an alpha-mannan degrading enzyme for reduction of redeposition, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

The compositions of the invention comprise a blend of DNase and an alpha-mannan degrading enzyme and effectively reduce or limit malodor of e.g. textiles or hard surfaces such as dishes.

One embodiment of the invention relates to the use of a composition comprising a DNase and an alpha-mannan degrading enzyme for reduction of malodor. One embodiment of the invention relates to the use of a cleaning composition comprising a DNase and an alpha-mannan degrading enzyme for reduction of malodor.

One embodiment of the invention relates to the use of a cleaning composition comprising a DNase and an alpha-mannan degrading enzyme for reduction of malodor when the cleaning composition is applied in e.g. laundry process. One embodiment of the invention relates to the use of a cleaning composition comprising a DNase and an alpha-mannan degrading enzyme for reduction of malodor on an item e.g. textile.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus, preferably Bacillus cibi, Bacillus horikoshii, Bacillus licheniformis, Bacillus subtilis, Bacillus horneckiae, Bacillus idriensis, Bacillus algicola, Bacillus vietnamensis, Bacillus hwajinpoensis, Bacillus indicus, Bacillus marisflavi or Bacillus luciferensis.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus and comprises one or both of the motif(s) [D/M/L][S/T]GYSR[D/N] (SEQ ID NO: 73) or ASXNRSKG (SEQ ID NO: 74).

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.

One embodiment of the invention relates to the use of a cleaning composition comprising a DNase and an alpha-mannan degrading enzyme for reduction of malodor, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

The compositions of the invention comprise a blend of DNase and alpha-mannan degrading enzyme and improve whiteness of textile. One embodiment of the invention relates to the use of a composition comprising a DNase and an alpha-mannan degrading enzyme for improve whiteness of an item e.g. a textile. One embodiment of the invention relates to the use of a cleaning composition comprising a DNase and an alpha-mannan degrading enzyme for improve whiteness. One embodiment of the invention relates to the use of a cleaning composition comprising a DNase and an alpha-mannan degrading enzyme improve whiteness when the cleaning composition is applied in e.g. laundry process. One embodiment of the invention relates to the use of a cleaning composition comprising a DNase and an alpha-mannan degrading enzyme improve whiteness on an item e.g. textile.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs:79, 80, 81, 82, 83, 84, 85, 86, 87, 88, 89, 90, 91, 92, 93, 94, 95, 96 or 97.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus, preferably Bacillus cibi, Bacillus horikoshii, Bacillus licheniformis, Bacillus subtilis, Bacillus horneckiae, Bacillus idriensis, Bacillus algicola, Bacillus vietnamensis, Bacillus hwajinpoensis, Bacillus indicus, Bacillus marisflavi or Bacillus luciferensis.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus and comprises one or both of the motif(s) [D/M/L][S/T]GYSR[D/N] (SEQ ID NO: 73) or ASXNRSKG (SEQ ID NO: 74).

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.

One embodiment of the invention relates to the use of a cleaning composition comprising a DNase and an alpha-mannan degrading enzyme for improve whiteness, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

The compositions of the invention comprise a blend of DNase and alpha-mannan degrading enzyme and effectively reduce or remove poly-organic stains comprising organic components, such as polysaccharides and DNA from surfaces such as textiles and hard surfaces e.g. dishes. One embodiment of the invention relates to the use of a cleaning composition comprising a DNase, an alpha-mannan degrading enzyme and at least one cleaning component for reduction or removal of biofilm and components of biofilm, such as DNA and polysaccharides, of an item, wherein the item is a textile or a hard surface.

One embodiment of the invention relates to the use of a cleaning composition comprising a DNase, an alpha-mannan degrading enzyme and at least one cleaning component for deep cleaning of an item, wherein the item is a textile or a surface.

One embodiment of the invention relates to the use of a composition comprising a DNase and an alpha-mannan degrading enzyme for reduction or removal of biofilm compounds such as DNA and polysaccharides of an item. One embodiment of the invention relates to the use of a cleaning composition comprising a DNase and an alpha-mannan degrading enzyme for reduction or removal of biofilm compounds such as DNA and polysaccharides of an item such as textile. One embodiment of the invention relates to the use of a cleaning composition comprising a DNase and an alpha-mannan degrading enzyme for deep cleaning e.g. reduction or removal of biofilm compounds such as DNA and polysaccharides when the cleaning composition is applied in e.g. laundry process.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus, preferably Bacillus cibi, Bacillus horikoshii, Bacillus licheniformis, Bacillus subtilis, Bacillus horneckiae, Bacillus idriensis, Bacillus algicola, Bacillus vietnamensis, Bacillus hwajinpoensis, Bacillus indicus, Bacillus marisflavi or Bacillus luciferensis.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the DNase is obtained from Bacillus and comprises one or both of the motif(s) [D/M/L][S/T]GYSR[D/N] (SEQ ID NO: 73) or ASXNRSKG (SEQ ID NO: 74).

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.

One embodiment of the invention relates to the use of a cleaning composition comprising a DNase and an alpha-mannan degrading enzyme for deep clean of an item, wherein the alpha-mannan degrading enzyme has at least 60%, at least 65%, at least 70%, at least 75%, at least 80%, at least 85%, at least 90%, at least 95%, at least 98% or 100% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

The invention further relates to a method of deep cleaning an item, wherein the item may be textile or hard surface preferably is a textile,

One embodiment of the invention relates to a method of cleaning e.g. deep cleaning an item, comprising the steps of:
a) contacting the item with a cleaning composition according to the invention; and
b) and optionally rinsing the item, wherein the item is preferably a textile.
   One embodiment of the invention relates to a method of cleaning on an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile.

One embodiment of the invention relates to a method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97.
   One embodiment of the invention relates to a method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 13.
   One embodiment of the invention relates to a method of cleaning e.g. deep cleaning an item, comprising the steps of:
a) contacting the item with a solution comprising an enzyme mixture comprising a DNase and a alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 65.
   One embodiment of the invention relates to a method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 66.
   One embodiment of the invention relates to a method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 67.
   One embodiment of the invention relates to a method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 0.1 to 15 wt %, preferably 1 to 30 wt % or preferably 1 to 60 wt % of at least one a surfactant; 0.5 to 20 wt %, preferably 1 to 40 wt % of at least one builder; and 0.01 to 10 wt %, preferably 1 to 20 wt % of at least one bleach component; and
b) and optionally rinsing the item, wherein the item is preferably a textile,
   wherein the alpha-mannan degrading enzyme has at least 60%, at least 65%, at least 70%, at least 75%, at least 80%, at least 85%, at least 90%, at least 95% or at least 98% sequence identity to the amino acid sequence shown in SEQ ID NOs: 79, 80, 81, 82, 83, 84, 85, 86, 87, 88, 89, 90, 91, 92, 93, 94, 95, 96 or 97 and wherein the is DNase has at least 60%, at least 65%, at least 70%, at least 75%, at least 80%, at least 85%, at least 90%, at least 95%, at least 98% or 100% sequence identity to the amino acid sequence shown in SEQ ID NO: 68.

### Definitions

Biofilm is produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, Staphylococcus aureus and Stenotrophomonas sp. In one aspect, the biofilm producing strain is Brevundimonas sp. In one aspect, the biofilm producing strain is Pseudomonas alcaliphila or Pseudomonas fluorescens. In one aspect, the biofilm producing strain is Staphylococcus aureus.

By the term “deep cleaning” is meant reduction, disruption or removal of components which may be comprised in organic matter, e.g. biofilm, such as polysaccharides, proteins, DNA, soil or other components present in the organic matter. In the context of the present invention organic matter is e.g. a poly-organic stain i.e. a stain comprising more than one organic component such as stains from body soiling e.g. skin cell debris, sebum, sweat, and biofilm, EPS, etc. which comprises several organic molecules such as polysaccharides, extracellular DNA (exDNA), mannan e.g. α-mannan, starch and proteins.

Cleaning component: The cleaning component e.g. the detergent adjunct ingredient is different to the DNase and alpha-mannan degrading enzyme enzymes. The precise nature of these additional cleaning components e.g. adjunct components, and levels of incorporation thereof, will depend on the physical form of the composition and the nature of the operation for which it is to be used. Suitable cleaning components e.g. adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning composition: The term “cleaning composition” refers to compositions that find use in the removal of undesired compounds from items to be cleaned, such as textiles. The cleaning composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzymes, the cleaning composition may contain one or more additional enzymes (such as amylases, lipases, cutinases, cellulases, endoglucanases, xyloglucanases, pectinases, pectin lyases, xanthanases, peroxidases, haloperoxygenases, catalases and mannanases, or any mixture thereof), and/or cleaning components e.g. detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides. Textile care benefits, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.”

The term “hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

The term “wash performance” is used as an enzyme's ability to remove stains present on the object to be cleaned during e.g. wash or hard surface cleaning.

The term “whiteness” is defined herein as a greying, yellowing of a textile. Loss of whiteness may be due to removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

By the term “malodor” is meant an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

The term “mature polypeptide” means a polypeptide in its final form following translation and any post-translational modifications, such as N-terminal processing, C-terminal truncation, glycosylation, phosphorylation, etc.

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

The term “variant” means a polypeptide having the activity of the parent or precursor polypeptide and comprising an alteration, i.e., a substitution, insertion, and/or deletion, at one or more (e.g., several) positions compared to the precursor or parent polypeptide. A substitution means replacement of the amino acid occupying a position with a different amino acid; a deletion means removal of the amino acid occupying a position; and an insertion means adding an amino acid adjacent to and immediately following the amino acid occupying a position.

A polypeptide having DNase or alpha-mannan degrading activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The invention may further relate to any of the following embodiments;

Paragraph 1. A cleaning composition comprising at least 0.001 ppm DNase, at least 0.001 ppm alpha-mannan degrading enzyme and a cleaning component, wherein the cleaning component is selected from
a. 1 to 40 wt % of at least one a surfactant;
b. 0.5 to 30 wt % of at least one builder; and
c. 0.1 to 20 wt % of at least one bleach component.

Paragraph 2. The cleaning composition according to paragraph 1, wherein the DNase comprises one or both of the motif(s) [D/M/L][S/T]GYSR[D/N] (SEQ ID NO: 73) or ASXNRSKG (SEQ ID NO: 74).

Paragraph 3. The cleaning composition according to paragraphs 1 or 2, wherein the DNase is selected from the group of polypeptides:
a) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 1,
b) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 2,
c) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 3,
d) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 4,
e) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 5,
f) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 6,
g) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 7,
h) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 8,
i) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 9,
j) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 10,
k) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 11,
l) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 12,
m) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 13,
n) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 14,
o) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 15,
p) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 16,
q) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 17,
r) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 18,
s) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 19,
t) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 20,
u) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 21,
v) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 22,
w) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 23,
x) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 24, and
y) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 25.

Paragraph 4. The cleaning composition according to paragraph 1, wherein the DNase comprises one or both of the motif(s) [V/I]PL[S/A]NAWK (SEQ ID NO: 75) or NPQL (SEQ ID NO: 76).

Paragraph 5. The cleaning composition according to paragraph 1 or 4, wherein the DNase is selected from the group of polypeptides:
a) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 26,
b) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 27,
c) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 28,
d) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 29,
e) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 30,
f) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 31,
g) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 32,
h) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 33,
i) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 34,
j) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 35,
k) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 36,
l) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 37, and
m) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 38.

Paragraph 6. The cleaning composition according to paragraph 1 wherein the DNase comprises one or both of the motif(s) P[Q/E]L[W/Y] (SEQ ID NO: 77) or [K/H/E]NAW (SEQ ID NO: 78).

Paragraph 7. The cleaning composition according to paragraphs 1 or 6, wherein the DNase is selected from the group of polypeptides:
a) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 39,
b) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 40,
c) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 41,
d) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 42,
e) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 43
f) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 44,
g) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 45,
h) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 46,
i) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 47,
j) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 48,
k) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 49,
l) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 50, and
m) a polypeptide having at least 60%, at least 65%, at least 70%, at least 75%, at least 80%, at least 85%, at least 90%, at least 95%, at least 98% or 100% sequence identity to the polypeptide shown in SEQ ID NO: 51.

Paragraph 8. The cleaning composition according to paragraph 1, wherein the DNase is selected from the group consisting of:
a) a polypeptide obtainable from Bacillus cibi having a sequence identity to the polypeptide shown in SEQ ID NO: 13 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity,
b) a polypeptide obtainable from Bacillus licheniformis having a sequence identity to the polypeptide shown in SEQ ID NO: 65 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity,
c) a polypeptide obtainable from Bacillus subtilis having a sequence identity to the polypeptide shown in SEQ ID NO: 66 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity,
d) a polypeptide obtainable from Aspergillus oryzae having a sequence identity to the polypeptide shown in SEQ ID NO: 67 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity,
e) a polypeptide obtainable from Trichoderma harzianum having a sequence identity to the polypeptide shown in SEQ ID NO: 68 of at least 60%, e.g., at least 65%, at least 70%, at least 75%, at least 80%, at least 85%, at least 90%, at least 95%, at least 96%, at least 97%, at least 98%, at least 99%, or 100% and which have DNase activity,

and combinations thereof.

Paragraph 9. The cleaning component of any of paragraphs 1 to 8 wherein the alpha-mannan degrading enzyme is selected from,
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 79 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 80 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 81 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 82 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 83 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 84 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 85 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 86 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 87 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 88 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 89 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 90 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 91 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 92 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 93 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 94 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 95 or a fragment thereof having alpha-mannan degrading activity;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 96 or a fragment thereof having alpha-mannan degrading activity; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 97 or a fragment thereof having alpha-mannan degrading activity.

Paragraph 10. The use of a composition according to any of the previous paragraphs for deep cleaning of an item, wherein the item is a textile or a surface.

Paragraph 11. A method of formulating a cleaning composition comprising adding a DNase, an alpha-mannan degrading enzyme and at least one cleaning component.

Paragraph 12. A kit intended for deep cleaning, wherein the kit comprises a solution of an enzyme mixture comprising a DNase and an alpha-mannan degrading enzyme.

Paragraph 13. A method of cleaning e.g. deep cleaning an item, comprising the steps of:
a) contacting the item with a solution comprising an enzyme mixture comprising a DNase and an alpha-mannan degrading enzyme; and a cleaning component, wherein the cleaning component is selected from 1 to 40 wt % of at least one a surfactant; 0.5 to 30 wt % of at least one builder; and 0.1 to 20 wt % of at least one bleach component; and
b) and optionally rinsing the item, wherein the item is preferably a textile.

### EXAMPLES

### Assays

### Assay I DNase Activity

DNase activity is determined on DNase Test Agar with Methyl Green (BD, Franklin Lakes, N.J., USA), which is prepared according to the manual from supplier. Briefly, 21 g of agar is dissolved in 500 ml water and then autoclaved for 15 min at 121° C. Autoclaved agar is temperated to 48° C. in water bath, and 20 ml of agar is poured into petri dishes with and allowed to solidify by incubation o/n at room temperature. On solidified agar plates, 5 μl of enzyme solutions are added and DNase activity is observed as colorless zones around the spotted enzyme solutions

### Assay II DNase Activity

DNase activity is determined by using the DNaseAlert™ Kit (11-02-01-04, IDT Intergrated DNA Technologies) according to the supplier's manual. Briefly, 95 μl DNase sample is mixed with 5 μl substrate in a microtiter plate, and fluorescence is immediately measured using a Clariostar microtiter reader from BMG Labtech (536 nm excitation, 556 nm emission).

### Assay III Reducing End Assay (Alpha-Mannanase Activity)

For estimating the mannose yield after substrate hydrolysis, a reducing end assay developed by Lever (Anal. Biochem. 47: 273-279, 1972) is used. The assay is based on 4-hydroxybenzoic acid hydrazide, which under alkaline conditions reacts with the reducing ends of saccharides. The product is a strong yellow anion, which absorbs at 405 nm.

Method. The hydrolysis reaction mixture is composed of 20 μL enzyme and 180 μL substrate dissolved in buffer. The substrate is alfa-1,6-mannan prepared as described elsewhere (Cuskin, Nature, 2015, 517, 165-169) at a concentration of 2 mg/mL. The buffer is 25 mM acetate, pH5.5, 50 mM KCl, 0.01% Triton X-100, 1 mM CaCl2. The reaction conditions are 30 minutes, 37° C., and 950 rpm. 4-Hydroxybenzhydrazide (PAHBAH) (Sigma, H9882) is diluted in PAHBAH buffer to a concentration of 15 mg/ml. PAHBAH buffer contains: 50 g/L K-Na-tartrate (Merck, 1.08087) and 20 g/L sodium hydroxide (Sigma, S8045). This PAHBAH mix is made just before usage. 70 μL PAHBAH mix and MiliQ water are mixed in a 96 well PCR plate (Thermo Scientific). Samples from hydrolysis experiment are added. Samples and MiliQ always reached the total volume of 150 μL, but the dilution of the sample differed. The plate is sealed with Adhesive PCR Sealing Foil Sheets (Thermo Scientific). Plates are incubated at 95° C. for 10 min, cooled down and kept at 10° C. for 1 min in PTC-200 Thermal Cycler (MJ Research). 100 μL sample is transferred to a 96 well microtiter plate, flat bottomed (Nunc™) and color development measured at 405 nm on a SpectraMax 190 Absorbance Microplate Reader (Molecular Devices). Results are compared to mannose standards, which had undergone the same treatment and dilution as the samples to which they were compared.

### Model Detergents

Model detergent A wash liquor (100%) is prepared by dissolving 3.33 g/l of model detergent A containing 12% LAS, 11% AEO Biosoft N25-7 (NI), 17.63% AEOS (SLES), 6% MPG, 3% ethanol, 3.33% TEA (triethanolamine), 2.75% cocoa soap, 2.75% soya soap, 1.7% glycerol, 1.75% sodium hydroxide, 2% sodium citrate, 1% sodium formate, 0.48% DTMPA and 0.46% PCA (all percentages are w/w (weight volume) in water with hardness 15 dH.

Triple-20 Nonionic Model Detergent (60% surfactant) is prepared by dissolving 3.33 g/I non-ionic detergent containing NaOH 0.87%, MPG (Monopropylenglycol) 6%, Glycerol 2%, Soap-soy 2.75%, Soap-coco 2.75%, PCA (Sokalon CP-5) 0.2%, AEO Biosoft N25-7(NI) 16%, Sodium formiate 1%, Sodium Citrate 2%, DTMPA 0.2%, Ethanol (96%) 3%, adjustment of pH with NaOH or Citric acid add water to 100% (all percentages are w/w (weight volume) in water with hardness 15 dH.

Model Detergent MC: A medical cleaning model detergent (model detergent MC) is prepared containing 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH is adjusted to 8.7 with phosphoric acid.

### Example 1

### Preparation of Biofilm Swatches

Biofilm swatches were made by growing Brevundimonas sp. on polyester swatches for two days.

The biofilm swatches were rinsed twice in water and dried for 1 h under flow and subsequently punched into small circles and stored at 4° C. for further use.

### Washing Experiment

Biofilm swatches punctures were placed in a deep well 96 format plate. The 96 well plate was placed in a Hamilton robot and subjected to a wash simulation program using the following conditions: Shaking speed: 30 sec at 1000 rpm. Duration of wash cycle: 30 minutes with shaking; temperature 30° C.; Volume of wash liquor (total): 0.5 ml per well. (490 wash liquor+10 uL sample). For wash performance assay, Model detergent A (3.3 g/L) dissolved in water hardness 15° dH was used. Soil was subsequently added to reach a concentration of 0.7 g soil/L (WFK 09V pigment soil). A 96 well plate was filled with each enzyme sample, and the program was started on the robot. The DNase (SEQ ID NO 13) was used in low dose (0.00001 ppm) to show synergy. The alpha-mannanase (alpha-mannan degrading enzyme) (SEQ ID NO 88) was tested in a dose of 0.2 ppm and 0.4 ppm. The blank consisted of biofilm swatches without any enzyme addition. After completion of the wash simulation cycle, the swatch punctures were removed from the wash liquor and dried on a filter paper. The dried swatch punctures were fixed on a sheet of white paper for scanning. The scanned picture was further used with the software colour-analyzer. Each sample will have an intensity measurement, from the colour analyzer software analysis, that will be used to calculate the delta intensity (remission), by subtracting the intensity of the blank, without enzyme. Values over 40 are visual for the human eye.

TABLE 1
Wash performance of alpha-mannanase (SEQ ID NO 88)
with and without DNase (SEQ ID No 13).

| | | | | Intensity | |
|---|---|---|---|---|---|
| | | | Delta | (Alpha- | |
| | | Intensity | intensity | mannanase + | Delta intensity (Alpha- |
| | | (individual) | (individual) | DNase) | mannanase + DNase) |
| 0.2 ppm | Alpha- | 241 | 31 | 271 | 61 |
| | mannanase | | | | |
| 0.4 ppm | Alpha- | 276 | 66 | | |
| | mannanase | | | | |
| 0.00001 ppm | DNase | 231 | 21 | | |
| | No enzyme, | 210 | 0 | | |
| | blank | | | | |

Conclusion: Instead of doubling the dose of alpha-mannanase from 0.2 ppm to 0.4 ppm, it was possible to get the same wash performance of 0.4 ppm alpha-mannanase by combining alpha-mannanase (0.2 ppm) with a small amount of DNase (0.00001 ppm).

1. A cleaning composition comprising a DNase, an alpha-mannan degrading enzyme and at least one cleaning component.

2. A cleaning composition according to claim 1, wherein the alpha-mannan degrading enzyme is of the GH family GH76, GH92 or GH99.

3. A cleaning composition according to claim 1, wherein the alpha-mannan degrading enzyme is a GH76 glycosyl hydrolase.

4. A cleaning composition according to claim 3, wherein the alpha-mannan degrading enzyme has at least 60%, sequence identity to the amino acid sequence shown in SEQ ID NOs: 79, 80, 81, 82, 83, 84, 85, 86, 89, 90, 91, 92, 93, 94, 95, 96 and 97.

5. A cleaning composition according to claim 1, wherein the alpha-mannan degrading enzyme is a GH92 glycosyl hydrolase.

6. A cleaning composition according to claim 5, wherein the alpha-mannan degrading enzyme has at least 60%, sequence identity to the amino acid sequence shown in SEQ ID NO: 87.

7. A cleaning composition according to claim 1, wherein the alpha-mannan degrading enzyme is a GH99 glycosyl hydrolase.

8. A cleaning composition according to claim 7, wherein the alpha-mannan degrading enzyme has at least 60%, sequence identity to the amino acid sequence shown in SEQ ID NO: 88.

9. A cleaning composition according to claim 1, wherein the DNase is obtained from bacteria or fungi.

10. A cleaning composition according to claim 1, wherein the DNase is obtained from Bacillus.

11. A cleaning composition according to claim 1, wherein the DNase comprises one or both of the motif(s) [D/M/L][S/T]GYSR[D/N] (SEQ ID NO: 73) or ASXNRSKG (SEQ ID NO: 74).

12. A cleaning composition according claim 9, wherein the DNase has at least 60% sequence identity to the amino acid sequence shown in SEQ ID NO: 13.

13. A cleaning composition according to claim 9, sequence identity to the amino acid sequence shown in SEQ ID NO: 65.

14. A cleaning composition according to claim 9, wherein the DNase has at least 60% sequence identity to the amino acid sequence shown in SEQ ID NO: 66.

15. A cleaning composition according to claim 9, wherein the DNase sequence identity to the amino acid sequence shown in SEQ ID NO: 67.

16. A cleaning composition according to claim 9, wherein the DNase sequence identity to the amino acid sequence shown in SEQ ID NO: 68.

17. A cleaning composition according to claim 1, wherein the amount of DNase in the composition is from 0.01 to 1000 ppm and the amount of alpha-mannan degrading enzyme is from 0.01 to 1000 ppm.

18. A cleaning composition according to claim 1, wherein the cleaning component is selected from surfactants, builders and bleach components.

19. (canceled)

20. A method of cleaning of an item, comprising the steps of:
a) contacting the item with a cleaning composition according to claim 1; and
b) optionally rinsing the item, wherein the item is preferably a textile.


SEQUENCE LISTING (97 sequences)

SEQ ID NO: 1 - protein, 182 aa, Bacillus sp.
LPPDLPSKSTTQAQLNSLNVKNEESMSGYSREKFPHWISQGDGCDTRQVILKRDADNYSGNCPVTSGKWYSYYDGITFNDPSQLDIDHVVPLAEAWRSGASSWSTAKREDFANDLNGPQLIAVSASSNRSKGDQDPSTWQPPRAGANCAYAKMWINTKYNWGLHLQSSEKTALQGMLNSCSY

SEQ ID NO: 2 - protein, 182 aa, Bacillus horikoshii
LPPGTPTKSEAQNQLNSLTVKSEGSMTGYSRDLFPHWSGQGNGCDTRQIVLQRDADYYTGTCPTTSGKWYSYFDGVIVYSPSEIDIDHIVPLAEAWRSGASSWTTEQRRAFANDLNGPQLIAVTASVNRSKGDQDPSTWQPPRAGARCAYAKWWINTKHRWNLHLQSSEKSSLQTMLNGCAY

SEQ ID NO: 3 - protein, 182 aa, Bacillus sp.
LPPGTPSKSEAQSQLNALTVKPEDPMTGYSRDHFPHWISQGNGCNTRQIVLQRDADYYSGACPVTTGKWYSYFDGVIVYSPSEIDIDHIVPLAEAWRSGASSWTTEKRRSFANDLNGPQLIAVTASVNRSKGDQDPSTWQPPRAGARCAYAKWWINTKHRWGLHLQSSEKSSLQSMLNGCAY

SEQ ID NO: 4 - protein, 182 aa, Bacillus sp.
LPPGTPSKSEAQSQLNALTVKPEDPMTGYSRDHFPHWISQGNGCNTRQIVLQRDADYYSGACPVTTGKWYSYFDGVIVYSPSEIDIDHIVPLAEAWRSGASSWTTEQRRSFANDLNGPQLIAVTASVNRSKGDQDPSTWQPPRAGARCAYAKWWINTKHRWGLHLQSSEKSSLQSMLNGCAY

SEQ ID NO: 5 - protein, 182 aa, Bacillus horikoshii
LPPGTPSKSEAQSQLNSLTVKSEDPMTGYSRDHFPHWSGQGNGCDTRQIVLQRDADYYSGNCPVTSGKWYSYFDGVIVYSPSEIDIDHVVPLAEAWRSGASSWTTEQRRSFANDLNGPQLIAVTASVNRSKGDQDPSTWQPPRAGARCAYAKWWINTKHRWNLHLQSSEKSALQTMLNGCVY

SEQ ID NO: 6 - protein, 182 aa, Bacillus horikoshii
LPPGTPSKSEAQSQLNSLTVKTEDPMTGYSRDLFPHWSGQGSGCDTRQIVLQRDADYFTGTCPTTSGKWYSYFDGVIVYSPSEIDVDHIVPLAEAWRSGASSWTTEQRRAFANDLTGPQLIAVTASVNRSKGDQDPSTWQPPRAGARCAYAKWWINTKHRWNLHLQSSEKSSLQTMLNGCAY

SEQ ID NO: 7 - protein, 182 aa, Bacillus sp.
LPPGTPSKSEAQSQLNALTVKAEDPMTGYSRNLFPHWNSQGNGCNTRQLVLQRDADYYSGNCPVTSGRWYSYFDGVVVTSPSEIDIDHIVPLAEAWRSGASSWTTEKRKEFANDLNGPQLIAVTASVNRSKGDQDPSTWQPPRAAARCGYAKWWINTKYRWDLSLQSSEKSSLQTMLNTCSY

SEQ ID NO: 8 - protein, 182 aa, Bacillus sp.
LPPGTPSKSQAQSQLNALTVKAEDPMTGYSRNLFPHWSSQGNGCNTRQLVLQRDADYYSGNCPVTSGRWYSYFDGVVVTSPSEIDIDHIVPLAEAWRSGASSWTTEKRREFANDLNGPQLIAVTASVNRSKGDQDPSTWQPPRVAARCGYAKWWINTKYRWDLSLQSSEKSSLQTMLNTCSY

SEQ ID NO: 9 - protein, 182 aa, Bacillus sp.
LPPGTPSKSEAQSQLTSLTVKPEDPMTGYSRDHFPHWISQGNGCNTRQIVLQRDADYYSGNCPVTTGKWYSYFDGVIVYSPSEIDIDHIVPLAEAWRSGASSWTAEQRRNFANDLNGPQLIAVTASVNRSKGDQDPSTWQPPRTGARCAYAKWWINTKYRWGLHLQSSEKSSLQSMLNGCAY

SEQ ID NO: 10 - protein, 183 aa, Bacillus sp.
AFPPGTPSKSTAQSQLNSLTVKSEGSMTGYSRDKFPHWISQGDGCDTRQLVLKRDGDYYSGNCPVTSGKWYSYYDGIAVYSPSEIDIDHIVPLAEAWRSGASGWTTEKRQNFANDLNGPQLIAVTASVNRSKGDQDPSTWQPPRSGSHCAYAKMWVNTKYRWGLHLQSAEKSALQSMLNACSY

SEQ ID NO: 11 - protein, 185 aa, Bacillus horneckiae
ASAFPPGTPSKSTAQSQLNSLTVKSEGSMTGYSRDKFPHWISQGDGCDTRQLVLKRDGDYYSGNCPVTSGKWYSYYDGITVYSPSEIDIDHIVPLAEAWRSGASGWTTEKRQSFANDLNGPQLIAVTASVNRSKGDQDPSTWQPPRSGSHCAYAKMWVNTKYRWGLHVQSAEKSALQSMLNACSY

SEQ ID NO: 12 - protein, 182 aa, Bacillus sp.
FPPEIPSKSTAQSQLNSLTVKSEDAMTGYSRDKFPHWISQGDGCDTRQMVLKRDADYYSGSCPVTSGKWYSYYDGITVYSPSEIDIDHIVPLAEAWRSGASSWTTEKRRNFANDLNGPQLIAVTASVNRSKGDQDPSTWQPPRSGARCAYAKMWVNTKYRWGLHLQSAEKSGLESMLNTCSY

SEQ ID NO: 13 - protein, 182 aa, Bacillus cibi [sequence omitted]

SEQ ID NO: 14 - protein, 182 aa, Bacillus sp.
FPPGTPSKSTAQSQLNSLTVKSEGSMTGYSRDKFPHWIGQGSGCDTRQLVLQRDADYYSGSCPVTSGKWYSYYDGVTFYDPSDLDIDHVVPLAEAWRSGASSWSTQKRKDFANDLSGPQLIAVSASSNRSKGDQDPSTWQPTRSGAACGYSKWWISTKHKWGLSLQSSEKNALQGMLNSCVY

SEQ ID NO: 15 - protein, 182 aa, Bacillus idriensis
LPPGTPSKSTAQSQLNALTVQTEGSMTGYSRDKFPHWISQGNGCDTRQVVLQRDADYYSGTCPVTSGKWYSYYDGVTLYNPSDLDIDHVVALAEAWRSGASSWTTDKREDFANDLSGTQLIAVSASTNRSKGDQDPSTWQPPRSGAACGYAKWWISTKYKWNLNLQSSEKTALQSMLNSCSY

SEQ ID NO: 16 - protein, 182 aa, Bacillus algicola
FPPGTPSKSEAQSQLNSLTVQSEGSMSGYSRDKFPHWIGQGNGCDTRQLVLQRDADYYSGDCPVTSGKWYSYFDGVTVYDPSDLDIDHMVPMAEAWRSGASSWSTQKREDFANDLSGPHLIAVTASSNRSKGDQDPSTWKPTRYGAHCGYAKWWINTKYVYDLTLQSSEKTELQSMLNTCSY

SEQ ID NO: 17 - protein, 182 aa, Enviromental sample J
LPPNIPSKADALTKLNALTVQTEGPMTGYSRDLFPHWSSQGNGCNTRHVVLKRDADSVVDTCPVTTGRWYSYYDGLVFTSASDIDIDHVVPLAEAWRSGASSWTSTKRQSFANDLNGPQLIAVSATSNRSKGDQDPSTWQPPRAGARCAYAKMWVETKSRWGLTLQSSEKAALQTAINACSY

SEQ ID NO: 18 - protein, 182 aa, Bacillus vietnamensis
FPPGTPSKSTAQSQLNALTVKSESSMTGYSRDKFPHWIGQRNGCDTRQLVLQRDADSYSGSCPVTSGSWYSYYDGVTFTDPSDLDIDHVVPLAEAWRSGASSWTTAKREDFANDLSGPQLIAVSASSNRSKGDQDPSTWQPPRSGAACGYSKWWISTKYKWGLSLQSSEKTALQGMLNSCIY

SEQ ID NO: 19 - protein, 182 aa, Bacillus hwajinpoensis
IPPGTPSKSAAQSQLDSLAVQSEGSMSGYSRDKFPHWIGQGNGCDTRQLVLQRDADYYSGDCPVTSGKWYSYFDGVQVYDPSYLDIDHMVPLAEAWRSGASSWSTQKREDFANDLDGPHLIAVTASSNRSKGDQDPSTWKPTRYSAHCGYAKWWINTKYVYDLNLQSSEKSALQSMLNTCSY

SEQ ID NO: 20 - protein, 182 aa, Paenibacillus mucilaginosus
LPPGTPSKSTAQSQLNSLTVKSESTMTGYSRDKFPHWTSQGGGCDTRQVVLKRDADYYSGSCPVTSGKWYSYYDGITVYSPSEIDIDHIVPLAEAWRSGASSWTTEKRQNFANDLGGPQLIAVTASSNRAKGDQDPSTWKPTRSGAHCAYAKWWINTKYRWGLHLQSSEKTALQSMLNTCSY

SEQ ID NO: 21 - protein, 182 aa, Bacillus indicus
TPPGTPSKSTAQTQLNALTVKTEGSMTGYSRDLFPHWISQGSGCDTRQVVLKRDADYYSGSCPVTSGKWYSYYDGVTFYDPSDLDIDHIVPLAEAWRSGASSWTTSKRQDFANDLSGPQLIAVSASTNRSKGDQDPSTWQPPRAGAACGYSKWWISTKYKWGLSLQSSEKTALQGMLNSCSY

SEQ ID NO: 22 - protein, 182 aa, Bacillus marisflavi
TPPVTPSKATSQSQLNGLTVKTEGAMTGYSRDKFPHWSSQGGGCDTRQVVLKRDADSYSGNCPVTSGSWYSYYDGVKFTNPSDLDIDHIVPLAEAWRSGASSWTTAQREAFANDLSGSQLIAVSASSNRSKGDQDPSTWQPPRAGAKCGYAKWWISTKSKWNLSLQSSEKTALQGMLNSCVY

SEQ ID NO: 23 - protein, 184 aa, Bacillus luciferensis
ASLPPGIPSLSTAQSQLNSLTVKSEGSLTGYSRDVFPHWISQGSGCDTRQVVLKRDADYYSGNCPVTSGKWYSYYDGVTVYSPSEIDIDHVVPLAEAWRSGASSWTTEKRQNFANDLNGPQLIAVTASSNRSKGDQDPSTWQPTRTGARCAYAKMWINTKYRWGLHLQSSEKSALQSMLNTCSY

SEQ ID NO: 24 - protein, 182 aa, Bacillus marisflavi
TPPVTPSKETSQSQLNGLTVKTEGAMTGYSRDKFPHWSSQGGGCDTRQVVLKRDADSYSGNCPVTSGSWYSYYDGVKFTHPSDLDIDHIVPLAEAWRSGASSWTTAQREAFANDLSGSQLIAVSASSNRSKGDQDPSTWQPPRAGAKCGYAKWWISTKSKWNLSLQSSEKTALQGMLNSCVY

SEQ ID NO: 25 - protein, 182 aa, Bacillus sp.
LPSGIPSKSTAQSQLNSLTVKSEGSMTGYSRDKFPHWISQGGGCDTRQVVLKRDADYYSGNCPVTSGKWYSYYDGISVYSPSEIDIDHVVPLAEAWRSGASSWTTTKRQNFANDLNGPQLIAVTASVNRSKGDQDPSTWQPPRYGARCAYAKMWINTKYRWDLNLQSSEKSSLQSMLDTCSY

SEQ ID NO: 26 - protein, 191 aa, Pyrenochaetopsis sp.
LPSPLLIARSPPNIPSATTAKTQLAGLTVAPQGPQTGYSRDLFPHWITQSGTCNTREVVLKRDGTNVVTNSACASTSGSWLSPYDGKTWDSASDIQIDHLVPLSNAWKSGAAAWTTAQRQAFANDLTHPQLVAVTGSVNESKGDDGPEDWKPPLASYYCTYASMWTAVKSNYKLTITSAEKSALTSMLATC

SEQ ID NO: 27 - protein, 190 aa, Vibrissea flavovirens
TPLPIIARTPPNIPTTATAKSQLAALTVAAAGPQTGYSRDLFPTWITISGTCNTRETVLKRDGTNVVVDSACVATSGSWYSPYDGATWTAASDVDIDHMVPLSNAWKSGASAWTTAQRQTFANDLTNPQLLAVTDNVNQAKGDSGPEDWKPSLTSYWCTYAKMWVKVKTVYDLTITSAEKTALTTMLNTC

SEQ ID NO: 28 - protein, 192 aa, Setosphaeria rostrata
APTSSPLVARAPPNVPSKAEATSQLAGLTVAPQGPQTGYSRDLFPHWITQSGTCNTRETVLKRDGTNVVTNSACASTSGSWFSPYDGATWTAASDVDIDHMVPLSNAWKSGAASWTTARRQAFANDLTNPQLLAVTDNVNQAKGDKGPEDWKPPLTSYYCTYSKMWIKVKSVWGLTITSAEKSALTSMLATC

SEQ ID NO: 29 - protein, 192 aa, Endophragmiella valdina
APVPGHLMPRAPPNVPTTAAAKTALAGLTVQAQGSQTGYSRDLFPHWITQSGTCNTREVVLKRDGTNVVTDSACAATSGTWVSPYDGATWTAASDVDIDHMVPLSNAWKSGAASWTTAQRQAFANDLTNPQLLAVTDNVNQSKGDKGPEDWKPPLTSYYCTYAKMWVKVKSVYSLTITSAEKTALTSMLNTC

SEQ ID NO: 30 - protein, 190 aa, Corynespora cassiicola
LPAPLVPRAPPGIPTTSAARSQLAGLTVAAQGPQTGYSRDLFPHWITQSGSCNTREVVLARDGTGVVQDSSCAATSGTWRSPFDGATWTAASDVDIDHMVPLSNAWKSGAASWTTSRRQAFANDLTNPQLIAVTDNVNQSKGDKGPEDWKPPLTSYYCTYAKMWVRVKSVYSLTITSAEKSALTSMLDTC

SEQ ID NO: 31 - protein, 192 aa, Paraphoma sp.
APAPVHLVARAPPNVPTAAQAQTQLAGLTVAAQGPQTGYSRDLFPHWITQSGACNTRETVLKRDGTGVVQDSACAATSGTWKSPYDGATWTAASDVDIDHMVPLSNAWKSGAASWTTARRQAFANDLTNPQLLAVTDNVNQAKGDKGPEDWKPPLTSYYCIYARMWIKVKSVYSLTITSAEKSALTSMLGTC

SEQ ID NO: 32 - protein, 186 aa, Monilinia fructicola
TPVPAPTGIPSTSVANTQLAALTVAAAGSQDGYSRDLFPHWITISGACNTRETVLKRDGTNVVVNSACAATSGTWVSPYDGATWTAASDVDIDHLVPLSNAWKAGASSWTTAQRQAFANDLVNPQLLAVTDSVNQGKSDSGPEAWKPSLKSYWCTYAKMWIKVKYVYDLTITSAEKSALVTMMDTC

SEQ ID NO: 33 - protein, 190 aa, Curvularia lunata
APAPLSARAPPNIPSKADATSQLAGLTVAAQGPQTGYSRDLFPHWITQSGTCNTRETVLKRDGTNVVTSSSCAATSGTWFSPYDGATWTAASDVDIDHVVPLSNAWKSGAASWTTARRQAFANDLTNPQLIAVTDSVNQAKGDKGPEDWKPPLSSYYCTYSKMWIKVKSVYGLTVTSAEKSALSSMLATC

SEQ ID NO: 34 - protein, 191 aa, Penicillium reticulisporum
LPAPEALPAPPGVPSASTAQSELAALTVAAQGSQDGYSRSKFPHWITQSGSCDTRDVVLKRDGTNVVQSASGCTITSGKWVSPYDGATWTASSDVDIDHLVPLSNAWKSGASGWTTAARQAFANDLTNPQLLVVTDNVNESKGDKGPEEWKPPLTSYYCTYAEMWVKVKSVYKLTITSAEKSALTSMLSTC

SEQ ID NO: 35 - protein, 191 aa, Penicillium quercetorum
LPAPEPAPSPPGIPSASTARSELASLTVAPQGSQDGYSRAKFPHWIKQSGSCDTRDVVLERDGTNVVQSSTGCTITGGTWVSPYDGATWTASSDVDIDHLVPLSNAWKSGASAWTTAQRQAFANDLTNPQLVAVTDNVNEAKGDKGPEEWKPPLTSYYCTYAEMWVKVKSVYKLTITSAEKSALSSMLNTC

SEQ ID NO: 36 - protein, 192 aa, Setophaeosphaeria sp.
LPAPVTLEARAPPNIPSTASANTLLAGLTVAAQGSQTGYSRDLFPHWITQSGTCNTRETVLKRDGTGVVTDSACASTSGSWYSVYDGATWTAASDVDIDHVVPLSNAWKSGAASWTTARRQSFANDLTNPQLIAVTDNVNQAKGDKGPEDWKPPLTSYYCTYAKMWVKVKSVYSLTITSAEKTALTSMLNTC

SEQ ID NO: 37 - protein, 192 aa, Alternaria sp.
LPAPVTLEARAPPNIPTTAAAKTQLAGLTVAAQGPQTGYSRDLFPHWITQSGTCNTRETVLKRDGTGVVTDSACASTSGSWFSVYDGATWTAASDVDIDHVVPLSNAWKSGAASWTTARRQSFANDLTNPQLIAVTDNVNQAKGDKGPEDWKPPLTSYYCTYAKMWVKVKSVYALTITSAEKTALTSMLNTC

SEQ ID NO: 38 - protein, 192 aa, Alternaria sp.
LPAPVTLEARAPPNIPTTAAAKTQLAGLTVAAQGPQTGYSRDLFPHWITQSGSCNTREVVLQRDGTGVVTDSACAATSGSWYSVYDGATWTAASDVDIDHMVPLSNAWKSGAASWTTARRQAFANDLTNPQLLAVTDNVNQAKGDKGPEDWKPPLTSYYCTYAKMWVKVKSVYALTITSAEKTALTSMLNTC

SEQ ID NO: 39 - protein, 186 aa, Trichoderma reesei
APLPAPPGIPSEDTARTQLAGLTVAVVGSGTGYSRDLFPTWDAISGNCNAREYVLKRDGEGVQVNNACEAQSGSWISPYDNASFTNASSLDIDHMVPLKNAWISGASTWTTAQREALANDVSRPQLWAVSASSNRSKGDRSPDQWKPPLTSFYCTYAKSWIDVKSYYKLTITSAEKTALSSMLDTC

SEQ ID NO: 40 - protein, 188 aa, Chaetomium thermophilum
APAPQPTPPGIPSRSTAQSYLNSLTVAASYDDGNYNRDLFPHWNTVSGTCNTREYVLKRDGSNVVTNSACQATSGTWYSPYDGATWTAASDIDIDHMVPLKNAWISGANTWSSSKRSSFANDINSPQLWAVTDSVNQSKGDKSPDKWKPPLTTFYCTYAKSWITVKYNYNLTITSAEKSALQNMINTC

SEQ ID NO: 41 - protein, 190 aa, Scytalidium thermophilum
LPAPAPMPTPPGIPSKSTAQSQLNALTVKASYDDGKYKRDLFPHWNTVSGTCNTREYVLKRDGVNVVTNSACAATSGTWYSPFDGATWTAASDVDIDHMVPLKNAWISGANNWTSTKRTQFANDINLPQLWAVTDDVNQAKGDKSPDKWKPPLTSFYCTYAKSWITVKYNYGLSITSAEKSALTSMINTC

SEQ ID NO: 42 - protein, 186 aa, Metapochonia suchlasporia
VPVPAPPGIPSTSTAKTLLAGLKVAVPLSGDGYSREKFPLWETIQGTCNAREFVLKRDGTDVKTNNACVAESGNWVSPYDGVKFTAARDLDIDHMVPLKNAWISGASQWTTERRKALANDITRPQLWAVSAHANRGKSDDSPDEWKPPLKTFWCTYAKSWVQVKSFYELTITDAEKGALAGMLDSC

SEQ ID NO: 43 - protein, 198 aa, Daldinia fissa
APAPIPVAEPAPMPMPTPPGIPSASSAKSQLASLTVKAAVDDGGYQRDLFPTWDTITGTCNTREYVLKRDGANVQVGSDCYPTSGTWTSPYDGGKWTSPSDVDIDHMVPLKNAWVSGANKWTTAKREQFANDVDRPQLWAVTDNVNSSKGDKSPDTWKPPLTSFYCTYASAYVAVKSYWGLTITSAEKSALSDMLGTC

SEQ ID NO: 44 - protein, 188 aa, Acremonium sp.
LPLQSRDPPGIPSTATAKSLLNGLTVKAWSNEGTYDRDLFPHWQTIEGTCNAREYVLKRDGQNVVVNSACTAQSGTWKSVYDGETTNSASDLDIDHMIPLKNAWISGAATWTTAQRTSFANDISSPQLWAVTAGVNRSKSDRSPDTWVPPLASFHCTYGKAWVQVKSKWALSITSAEKSALTGLLNKC

SEQ ID NO: 45 - protein, 182 aa, Acremonium dichromosporum
IPPGIPSEATARSLLSSLTVAPTVDDGTYDRDLFPHWSSVEGNCNAREFVLRRDGDGVSVGNDCYPTAGTWTCPYDGKRHSVPSDVSIDHMVPLHNAWMTGASEWTTAEREAFANDIDGPQLWAVTSTTNSQKGSDAPDEWQPPQTSIHCKYAAAWIQVKSTYDLTVSSAEQAALEEMLGRC

SEQ ID NO: 46 - protein, 188 aa, Sarocladium sp.
VPIPLPDPPGIPSSSTANTLLAGLTVRASSNEDTYNRDLFPHWVAISGNCNAREYVLRRDGTNVVVNTACVPQSGTWRSPYDGESTTNASDLDIDHMVPLKNAWISGAASWTTAKRQDFANDVSGPQLWAVTAGVNRSKGDKSPDSWVPPLASFHCTYARSWIQVKSSWALSVTSAEKAALTDLLSTC

SEQ ID NO: 47 - protein, 186 aa, Metarhizium sp.
VPVPAPPGIPTASTARTLLAGLKVATPLSGDGYSRTLFPTWETIEGTCNAREFVLKRDGTDVQTNTACVAQSGNWVSPYDGVAFTAASDLDIDHMVPLKNAWISGASQWTTDKRKGLANDITRPQLWAVSAHANRAKGDSSPDEWKPPLKTFWCTYARSWVQVKSYYALTITDAEKGALSGMLDSC

SEQ ID NO: 48 - protein, 188 aa, Acremonium sp.
APIAVRDPPGIPSASTANTLLAGLTVRASSNEDSYDRNLFPHWSAISGNCNAREFVLERDGTNVVVNNACVAQSGTWRSPYDGETTGNASDLDIDHMVPLKNAWISGASSWSTTRRQEFANDVSGPQLWAVTAGVNRSKGDRSPDSWVPPLASFHCTYAKSWVQVKSSWSLSVTSAEKAALSDLLGTC

SEQ ID NO: 49 - protein, 186 aa, Isaria tenuipes
APVPEPPGIPSTSTAQSDLNSLQVAASGSGDGYSRAEFPHWVSVEGSCDSREYVLKRDGQDVQADSSCKITSGTWVSPYDATTWTNSSKVDIDHLVPLKNAWISGASSWTKAQRQDFANDIKRPQLYAVSENANRSKGDRSPDGWKPPLKSFYCTYAKSWVAVKSYYKLTITSAEKSALGDMLDTC

SEQ ID NO: 50 - protein, 184 aa, Scytalidium circinatum
APPGIPSASTASSLLGELAVAEPVDDGSYDRDLFPHWEPIPGETACSAREYVLRRDGTGVETGSDCYPTSGTWSSPYDGGSWTAPSDVDIDHMVPLKNAWISGASEWTTAEREAFANDIDGPQLWAVTDEVNQSKSDQSPDEWKPPLSSFYCTYACAWIQVKSTYSLSISSAEQAALEDMLGSC

SEQ ID NO: 51 - protein, 186 aa, Metarhizium lepidiotae
VPVPAPPGIPTASTARTLLAGLKVATPLSGDGYSRTLFPTWETIEGTCNAREFVLKRDGTDVQTNTACVAESGNWVSPYDGVSFTAASDLDIDHMVPLKNAWISGASQWTTDKRKDLANDITRPQLWAVSAHANRSKGDSSPDEWKPPLQTFWCTYSKSWIQVKSHYSLTITDAEKGALSGMLDSC

SEQ ID NO: 52 - protein, 226 aa, Thermobispora bispora
LDIADGRPAGGKAAEAATGTSPLANPDGTRPGLAAITSADERAEARALIERLRTKGRGPKTGYEREKFGYAWADSVDGIPFGRNGCDTRNDVLKRDGQRLQFRSGSDCVVISMTLFDPYTGKTIEWTKQNAAEVQIDHVVPLSYSWQMGASRWSDEKRRQLANDPLNLMPVDGATNSRKGDSGPASWLPPRREIRCAYVVRFAQVALKYDLPVTTADKETMLQQCS

SEQ ID NO: 53 - protein, 191 aa, Sporormia fimetaria
LPAPVLEKRTPPNIPSTSTAQSLLSGLTVAPQGSQTGYSRDLFPHWITVSGTCNTRETVLKRDGSNVVTDSACASVSGSWYSTYDGATWTAASDVDIDHVVPLSNAWKSGAASWTTARRQAFANDLTNPQLIAVTDNVNQAKGDQGPESWKPPLTSYYCTYAKMWVKVKSVYSLTVTSAEKSALSSMLGTC

SEQ ID NO: 54 - protein, 193 aa, Pycnidiophora cf.dispera
LPAPAPVLVAREPPNIPSTSSAQSMLSGLTVKAQGPQDGYSRDLFPHWITISGTCNTRETVLKRDGTNVVTNSACASTSGSWYSPYDGATWTAASDVDIDHIVPLSNAWKSGAASWTTSRRQQFANDLTNPQLIAVTDSVNQAKGDKGPEDWKPSRTSYHCTYAKMWIKVKSVYSLTVTSAEKSALTTMLNTC

SEQ ID NO: 55 - protein, 199 aa, Enviromental sample D
DTDPEPVAGSALEALAGLEVKGPGPDTGYERALFGPPWADVDGNGCDTRNDILARDLTDLTFSTRGDVCEVRTGTFDDPYTGETIDFRRGNATSAAVQIDHVVPLLDAWRKGARAWDDETRRQFANDPLNLLASDGPANQSKGARDASAWLPPNHAFRCPYVARQIAVKAAYELSVTPSESEAMARVLADCPAEPLPAG

SEQ ID NO: 56 - protein, 199 aa, Enviromental sample O
DDEPEPARGSALEALARLEVVGPGPDTGYERELFGPAWADVDGNGCDTRNDILARDLTDLTFSTRGEVCEVRTGTFQDPYTGETIDFRRGNATSMAVQIDHVVPLMDAWRKGARAWDDETRRQFANDPLNLLASDGPANQSKGARDASAWLPPNHAFRCPYVARQIAVKTAYELSVTPSESEAMARVLEDCPAEPVPAG

SEQ ID NO: 57 - protein, 186 aa, Clavicipitaceae sp.
VPVPAPPGIPSTSTAKTLLAGLKVATPLSGDGYSRDKFPTWETIQGTCNAREFVIKRDGTDVKTNSACVAESGNWVSPYDGVKFTAARDLDIDHMVPLKNAWISGASQWTTEQRKALANDITRPQLWAVSAHANRGKSDDSPDEWKPPLKTFWCTYAKSWVQVKSFYKLTITDTEKGALAGMLDTC

SEQ ID NO: 58 - protein, 187 aa, Westerdykella sp.
FPAPASVLEARAPPNIPSASTAQSLLVGLTVQPQGPQDGYSRDLFPHWITISGTCNTRETVLKRDGSNVVTNSACAATSGTWYSPYDGATWTSASDVDIDHLVPLSNAWKSGAASWTTAKRQQFANDLTNPQLLAVTDRVNQAKGDKGPEAWKPSLASYHCTYAKMWVKVKSKDVRLTGNWTKDDGW

SEQ ID NO: 59 - protein, 194 aa, Humicolopsis cephalosporioides
APTPAPVELERRTPPNIPTTASAKSLLAGLTVAAQGPQTGYSRDLFPHWITISGSCNTRETVLKRDGTGVVTDSACASTAGSWYSPYDGATWTAASDVDIDHMVPLSNAWKSGAAQWTTARRQDFANDLTNPQLFAVTDNVNQEKGDKGPEDWKPSLTSYYCTYAKAWVKVKSVWALTITSAEKSALTTMLNTC

SEQ ID NO: 60 - protein, 190 aa, Neosartorya massa
IPAPVALPTPPGIPSAATAESELAALTVAAQGSSSGYSRDLFPHWISQGGSCNTREVVLARDGSGVVKDSNCYPTSGSWYSPYDGATWTQASDVDIDHVVPLANAWRSGASKWTTSQRQAFANDLTNPQLMAVTDNVNQAKGDDGPEAWKPPLTSYYCTYAKMWVRVKYVYDLTITSAEKSALVSMLDTC

SEQ ID NO: 61 - protein, 191 aa, Roussoella intermedia
APTPALLPRAPPNIPSTATAKSQLAALTVAAQGPQDGYSRDLFPHWITQSGSCNTREVVLKRDGTNVVQDSSCAATSGTWVSPFDGATWTAASDVDIDHLVPLSNAWKSGAASWTTARRQSFANDLTNPQLLAVTDEVNQAKGDKGPEAWKPPLASYHCTYAKMWVKVKSTYSLTITSAEKSALTTMLNTC

SEQ ID NO: 62 - protein, 191 aa, Pleosporales
LPTPSLVKRTPPNIPSTTSAKSLLAGLTVAAQGPQDGYSRDLFPHWITISGTCNTRETVLKRDGTNVVTDSACASTSGSWYSTYDGATWTAASDVDIDHVVPLSNAWKSGAASWTTARRQSFANDLTNPQLIAVTDSVNQSKGDKGPESWKPPLTSYHCTYAKMWVKVKDVYSLTVTSAEKSALTTMLNTC

SEQ ID NO: 63 - protein, 192 aa, Phaeosphaeria sp.
LPAPIHLTARAPPNIPSASEARTQLAGLTVAAQGPQDGYSRDLFPHWITQSGTCNTRETVLKRDGTNVVTNSACASTSGSWFSPYDGATWTAASDVDIDHMVPLSNAWKSGAASWTTARRQAFANDLTNPQLLAVTDNVNQAKGDKGPEDWKPPLTSYYCTYARMWVKVKSVYALTVTSAEKSALTSMLGTC

SEQ ID NO: 64 - protein, 189 aa, Didymosphaeria futilis
LPTPNTLEARAPPNIPSTSAAQSQLSALTVAAQGPQTGYSRDLFPHWITQSGTCNTRETVLKRDGTNVLTDSACASTSGSWKSPYDGATWTAASDVDIDHVVPLSNAWKSGAASWTTARRQSFANDLTNPQLIAVTDNVNQAKGDKGPEDWKPPLTSYYCTYAKMWVKVKSVYSLTITSAEKSALTMLA

SEQ ID NO: 65 - protein, 109 aa, Bacillus licheniformis
ARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 66 - protein, 110 aa, Bacillus subtilis
ASSYDKVLYFPLSRYPETGSHIRDAIAEGHPDICTIDRDGADKRREESLKGIPTKPGYDRDEWPMAVCEEGGAGADVRYVTPSDNRGAGSWVGNQMSSYPDGTRVLFIVQ

SEQ ID NO: 67 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 68 - protein, 188 aa, Trichoderma harzianum
APAPMPTPPGIPTESSARTQLAGLTVAVAGSGTGYSRDLFPTWDAISGNCNAREYVLKRDGEGVQVNNACESQSGTWISPYDNASFTNASSLDIDHMVPLKNAWISGASSWTTAQREALANDVSRPQLWAVSASANRSKGDRSPDQWKPPLTSFYCTYAKSWIDVKSFYKLTITSAEKTALSSMLDTC

SEQ ID NO: 69 - protein, 5 aa, Artificial
  Motif
  MISC_FEATURE (1)..(1): Xaa = Thr (T) or Asp (D) or Ser (S)
  MISC_FEATURE (2)..(2): Xaa = Gly (G) or Asn (N)
XXPQL

SEQ ID NO: 70 - protein, 5 aa, Artificial
  Motif
  MISC_FEATURE (1)..(1): Xaa = F (phe) or L (Leu) or Y (Tyr) or I (Ile)
  MISC_FEATURE (3)..(3): Xaa = N (Asn) or R (Arg)
  MISC_FEATURE (5)..(5): Xaa = L (Leu) or I (Ile) or P (Phe) or V (Val)
XAXDX

SEQ ID NO: 71 - protein, 4 aa, Artificial
  Motif
  MISC_FEATURE (2)..(2): Xaa= Asp (D) or Asn (N)
  MISC_FEATURE (4)..(4): Xaa= Ala (A) or Arg (R)
CXTX

SEQ ID NO: 72 - protein, 4 aa, Artificial
  Motif
  MISC_FEATURE (1)..(1): Xaa = Asp (D) or Gln (Q)
  MISC_FEATURE (2)..(2): Xaa = Ile (I) or Val (V)
XXDH

SEQ ID NO: 73 - protein, 7 aa, Artificial
  Motif
  MISC_FEATURE (1)..(1): Xaa = Asp (D) or Met (M) or Leu (L)
  MISC_FEATURE (2)..(2): Xaa = Ser (S) or Thr (T)
  MISC_FEATURE (7)..(7): Xaa = Asp (D) or Asn (N)
XXGYSRX

SEQ ID NO: 74 - protein, 8 aa, Artificial
  Motif
  MISC_FEATURE (3)..(3): Xaa = any amino acid
ASXNRSKG

SEQ ID NO: 75 - protein, 8 aa, Artificial
  Motif
  MISC_FEATURE (1)..(1): Xaa = Val (V) or Ile (I)
  MISC_FEATURE (4)..(4): Xaa = Ser (S) or Ala (A)
XPLXNAWK

SEQ ID NO: 76 - protein, 4 aa, Artificial
  Motif
NPQL

SEQ ID NO: 77 - protein, 4 aa, Artificial
  Motif
  MISC_FEATURE (2)..(2): Xaa = Gln (Q) or Glu(E)
  MISC_FEATURE (4)..(4): Xaa = Trp (W) or Tyr (Y)
PXLX

SEQ ID NO: 78 - protein, 4 aa, Artificial
  Motif
  MISC_FEATURE (1)..(1): Xaa=Lys (K) or His (H) or Glu (E)
XNAW

SEQ ID NO: 79 - protein, 353 aa, Paenibacillus glycanilyticus
ATSVIWQERAEEAQLELTKSFWDDKRGLYNNAAPCIAQLCTDPFNYWWLAHAVDALVDGYERSGDERYAEQIAKLHQGLLDRNAGVMINDYYDDMEWMALAWLRAYDATKDEKYKQEALELWEEIKGGWNEEMGGGIAWRKEQLDYKNTPANAPAAILAARLYGHFHNGEDLAWAKKIYDWQKETLVDPDTGLVWDGINRTGDGNIDKEWRFTYGQGVFIGAGVELFRATEDKAYLEDARRTAAHLKEAFLSPATGMLPSEGDGDGGLFKGVLIRYLGELIAADPDEPERKEWIGMLETNANSLWQYGKAEDKAVFSNSWAEAPDTIVQLSTELSGIMLLEQMAVLEKNGQLQ

SEQ ID NO: 80 - protein, 349 aa, Bacillus acidicola
ASYHKKNVERALASYELMQKYFYQPSVKLYTEEFPNVIGNSYSYLWPFSQAMAATTDVSRLPKIGKNYVFDRNDRLDGLKLYWNNGTNPAGYDSYVRPPLGQGGDKFYDDNDWIALNLIKLYQLTGDQAVLERVKDIFKLEVYGWDNDPSHPYRGGIFWTQASWSQDRNTISNAPLAQIGLYLYQITRDKSYFDWAKKAYDWVNNSMLAPNGLYWDHVDLKGNIDKTQWTYNQGMMIGANVLFYKTTGNETYLDLAKSIANKAIQYYGDVLLYNNPPEFNAIFFENLQLLDSINHNNIYRKYIQSYADQMWDTERNTETGLFQRDNQNPVPLIEQAAIVEIYSNLAYQK

SEQ ID NO: 81 - protein, 350 aa, Bacillus sp.
ASHQKKNTLRSITTYESLQKYFYQPSAKLYTEEYPREGGNPYSYVWPFSRAMAATIDMTRIPKIGKAYVSDRNERLEGLKLYWNNETNPAGYDSYVRPPVGQGGDKFYDDNDWIALNFLKLYQLTGDHSALKRAKDIFKLEVLGWDNDPSHPYPGGVFWTQAPWSQDRNTISNAPVAQIGLYLYQITGDKYYFDWAKKTYDWVNNSMLAPNGLYWDHVDLKGNIEKTQWTYNQGMMIGANVLFYKTTGDKMYLERAESLAEKSIQYYGDTQLYKNPPEFNAIFFENLQLLYSVKHNNEYRKYTQAYADKMWDTVRNPKTGLFQRDPQKPVPLIEQAAMVEIYANLAYKKQ

SEQ ID NO: 82 - protein, 479 aa, Paenibacillus sp.
AAFTAANADTAMNSFVSTFYDPVAKYFYTNSDHLIHSEHAHGPDGGLYTDFWWEAQLWETVMDAYERTGSSTYRAMIDDVYTGFNAKYPDMMANDFNDDLGWWALACMRAYELTGTDEYRNRASFLFDQIWGDWDSTYGGGIWWKRDGTSPQKNMATNAPMVMTAIKLKNATGNNDYLTKAQSIYSWIQSRLVSGSKINDHVEGSGSGTVVDWDFTYNYGTYLGAALALNQATGNASYLTDANTAAAYVMDKMTLSYSLMYEGENDSPGFRMVFARNLNQLRKATGNASYLNFLQQNATQAFNHRRASDGIIDSDWTAPARSGYIQSIAAAAGASILQLVPADNYTGPIAGNGTYEAENARRYGINNESSQPGFSGRGYTAGWNTDNTKIVFHVNQNSASTRTISFRYTAAGGNAGRYVKVNGTVVSNNLIFNGTSSWSAWNTVTLNVPLNAGYNSIELGFDSTKGNSNYLNVDKMTGL

SEQ ID NO: 83 - protein, 489 aa, Paenibacillus sp
FTAANANDAMQAFINVFYDPTAKYFYTNSDHQIHTHAHGPNGGLYTDFWWEAQLWETVMDAYERTGNATYRTMIDDIYTGFNAKYPDMMQNVFNDDLGWWAQAALRAYELTGTAEYRNRGSFLFDKIYEEWDTSYYGGGIWWRRDAHNPNVSSNAQKNVATNAPMVITAVKLYQATGDSAYLTKATQIYNWVKTKLVGSGGKINDHLEGPGAGTLIDWDFSYNYGNYLGAAVSLYQATGNSAYITDANTAATYAINNLVSAQTLMYEGENDAAGFKMIFARNLNRLRVIGGQSQYLNFLQQNATQAWNHRRTSDNIIGSDWLRPTGSGYIQSLAAAAGVSILQLTPPDNYTGYIAGNGAYEAENAQRTLVSGGGMINESTQGGYTGRGYVAGWNTTGTSLNFYVNQNTAGNRTITFRYAAGAGNASRYVRVNGVYVANNLSFSGTSGWGSWNTVSVTVPLNAGSNTIQLGYDSSRGNSNFLNVDILSGL

SEQ ID NO: 84 - protein, 348 aa, Paenibacillus sp.
ERLENYNNHAEWAEEKLIEHYWNENGKLMNNAYPYSKEREESLNYWWKAHAVDAMMDGYERTGDAAYTDRAEGIVKSIIKKNGSLFNEFYDDMEWLALAALRLYDATKSETVKGYVLKLWNDIKTAWWEDELGGMAWKKDQRDSRNACSNGPAAILAARMYEYFGDKEDLEWAKKIYDWQKKNLVDPESGLVYDGLKLEENGDLNVNKNWIFTYNQGTYIGAAVELYKHTNDKTYLADAEKTAASAMKYHTVAETGMMKEEGTGDGGLFKGIFIRYLIHLYEVNESVQIKEMIFHNADTLLSKGSTKEIGLFGPAWDMPHQEPLDISVQLSGVFLIEAAAKIERLDSE

SEQ ID NO: 85 - protein, 538 aa, Microbial community
YTASDGDTAMRAFNDTFWDPNAKMFWKDSKREKHQDFWVEAELWELVMDAYQHTSDPALKAELKTQIDDVYDGTVAKYGQDWTNNPFNDDIMWWAMGSARAYQITGNPRYLKAAKDHFDFVYDTQWDEEFASGGIWWLNSDHNTKNACINFPAAEAALYLYDITKDEHYLNTATKIFRWGKTMLTDGNGKVFDRIEVEHGAVPDATHYNQGTYIGAAVGLYEATGNAVYLDDAVKAAKFTKNHLVDSNGVLNYEGPNGDLKGGKTILMRNLAYLQKTLDETGQHPEFSAEFDEWLAFNVEMAWSHRNPDHIVDGNWAGQLLSGTYESWSSAAAVQALNVIKPMEAELRYAVKNPFEKIEAERYNIGAGFVLEGSFEGSLQLGGIQHGSYAAYKNVDFGSDGAIGFIARASSGTGGGQIEIRLDSKDGPKVGTLNVEGTGDWNHYIDAVTLLKDDQGAPSTITGVHDVYLVFTKTNDDYLFNLNWVQFTTTDPTETDAYAKLKAGNYDSSEGLSKHAEFGYLDGIHHNAYASYEGIDFG

SEQ ID NO: 86 - protein, 542 aa, Microbial community
FEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLISAYTILQAEHFASSDGLSINSTGQYADGIQNTAYASYENIDFG

SEQ ID NO: 87 - protein, 1378 aa, Bacillus novalis
SSDSTSASKTDFFSSFEKSDLQLTWTNTVETDANGKKMSSGIDGNVKRDLILGDITDKVVQVTASANNPPNEIDSKLIDGDPTTKWLAFEPTANIVLKLAEPVAVVKYALTSANDAKGRDPKNWTLYGSLDGTNWTAVDTREGEDFKDRFQRNMYDLKNTTKYLYYKLDITKNAGDSITQLAEISLSDGIEVPAPPPGDMKSLIGKGPTSSYTAKTNVGWTGLGALNYSGTHLSDGRAYSYNKLYDVDILVTPATELSYFIAPEFTDKNHNDYSSTYVSVDLAFSDGTYLHDLKAVDQYGVGLNPKDQGDSKYLYVNQWNTIKSTIGSVAAGKTIKRILVAYDNPKGPGAFRGSIDDIKIDGKPVQKAFGSPIDYVNILRGTQSNGSFSRGNNFPAVAIPHGFNFWTPTTNAGSSWIYQYHESNSVNNLPQIQAFSVSHEPSPWMGDRQTFQVMPSASTAATPNANRDSRALEFNHANEIAQPHYYSVKFENGIRTEMTPTDHAAMFKFTFTGATSNLIFDNVNNNGGLTIDAKSGEITGYSDVKSGLSTGATRLFVYAAFDKPVIKSGKLTGESRNNVTGYVRFDTSKDEDKVVTMKIATSLISVEQAKKNLEQEIGLNDTFEGLKEKAKTEWNKKLGIIEVEGASEDQLVTLYSNLYRLFLYPNSAFENVGTTTDPVYKYASPYSAATGQDTATTTGAKIVDGKTYVNNGFWDTYRTAWPAYSLLTPTFAGELIDGFVQQYRDGGWIARWSSPGFANLMPGTSSDVAFADAYLKGVTNFDVQSFYQSAIRNAEAVSPNAGTGRKGLTTSIFDGYTNTSTGEGLAWAMDGYINDFGIANLAKALKEKGDKSDPYYANYAADYQYFLNRAQNYVHMFNPSIEFFNGRTANGAWRSTPDNFNPAVWGSDYTETNGWNMAFHVPQDGQGLANLYGGKEGLATKLDQFFSTSETGLFPGSYGGTIHEMREARDVRMGMYGHSNQPSHHIAYMYDYAGQPWKTQEKVREALNRLYIGSAIGQGYSGDEDNGEMSAWYILSAMGFYPLKMGTPEYAIGAPLFKKATIHLENGKSIVINAPNNSKENKYVQSMKVNGKAYAKTSILHADIANGAVIDFEMGSKPSKWGSGDQDILQSITPGSTDGTSLSPLPLRDVTDRLIAAEKGAVTVSDEGNGQLLFDNTSNTQLSMKSKTPSIVYQFKEGKQNVKMYTLTSSKASQNEDPKSWVLKGSNDGKSWSVLDQRKNETFQWRQYTRAFTIQHPGKYSQYKLEITENAGAEVTTLAELELLGYDDVTNSYQAVYELMEQFKQSKDLTGPMAVQLNNSLTTSLDHFKKDHKDQAIKHLEDFLKHLNNKGLQDRISSKAKGVLSADANQLIVLLARD

SEQ ID NO: 88 - protein, 342 aa, Chryseobacterium sp.
QKETALRDKVQIFYYGWYGNQQTDGSLQHWNHEIIPHWSNPKWNNLGHYKGGNDIGANFYPGLGNYSSNDKKIIKKHMQMMKDSGVGVVVISWLGKDSFTDKSVMQYLDIAQQFNLKIAFHIEPFYKTITELRDQLSYLVEKYSQHPAFYKKDGKPMYYVYDSYKIAPEEWSKLLSENGEKTVRNTKLDALYIGLWVEKNDSEFFNKSGFDGFYTYFASEGFVFGSTTSNWKDMAQYAKDHHLIFIPCVGPGYSDTRIRPWNEANFKSRDNGKYYEKMFDAATKVNPEFIGITSFNEWHEGTQIEPAIPKKIDNFIYEDYGKDPWMYIKETKRLTDKFLKGK

SEQ ID NO: 89 - protein, 369 aa, Aspergillus aculeatus
TNYTAMTRAVTALNTLQTYYNPTTGIWNTCGWWNGANCLTTLANLSLKNSTVNDTATGVFENTFRVATNTNPYPARGIDADYTAANGTAYTISGQPTGAANASLWLDGSYDDDMWWGMAWVAAYDVTGVTDYLDLAEGVFYHLSRAWPSLCGNGGLDSDYTHVYVGAISNELFLALGASLANRVATNSSREYYLDWAKRQWAWFESSGLINANHTINDGLTGACTNNGMTVWSYNQGVILGGLVELHRATGSHASNSTYLTAAGKIAQGAIAALADEDDVIHESCEPDACDSNETQFKGIFIRNLKVLQGVAPNETYARVINASAASLWANDRTDATGFGIDWSGPVDAATVNASTQSSALDALVAAIW

SEQ ID NO: 90 - protein, 432 aa, Aspergillus aculeatus
IDLDINNEQSIKDAAATAAFNTMQHYNGNKTGEIPGVIPSLWGEGGVLFNLMIQYWYFTGDASYNPAVSQGMYWQIGDDDYMPSNWSSQIGNDDQMAWGLAAMTAAELDYPQDVNQTSWLTLAEGVFNTQVARWDTSNCGGGLRWQIWPFESGYTQKNAISNGGLFQLSARLARYTHNQTYADWADKIWDWSASVPLLNNKTWSIADSTNVDNGCTTQGNNQWTANYGPYISGAAYMYNYTNGQNSKWKSGLDGLLNVSFETFFPEKYGGLILSEILCEPAEVCNSLEDTYKGTFVSDLALASLVAPYISSEVSSRLQASAVGAAKQCTGGNNQTLCGRRWYSDEWDGTDGREEQLSATSIFFANMAGFTGKGVATAAAAVDQTTGSAGTNGTSTNGTGVPVALNGAGRSSQGQFGVATAGVLAGLVLLLVL

SEQ ID NO: 91 - protein, 384 aa, Aspergillus aculeatus
LQLDLNDEQSIKNAAATAAYNMMSYYHGNESGQIPGKLVDTWWEGGAMFMTLIQYWYWTGDTSYNAVTTEGMLWQKGQNDYFPANYSNYLGNDDQVFWGLAAMTAAELNYPEEDGQPSWLSLAQGVFNTQVPRWDTTSCQGGLRWQLWPYQAGYTTKNAISNGGLFQLAARLGRYTNNETYSNWAEKIYDWMATTPLLREDQWSIADTTTTQTECKDHGDLQWTYNYGTYISGAAYMYNHTNGGDKWKKALDGLLGTTLQKFFPQEFGGNIMSEISCEPNMMCDRNQDCFKGFLSSWLTFTTTIAPHTAGEIIPKIQQSALAAAKQCSGGKSGTECGRRWHQATWDGETSLESDMSALSVFSSTMIAHKGQEQSHQGPLTSETG

SEQ ID NO: 92 - protein, 380 aa, Aspergillus aculeatus
IDIDISSESSIKAAASKTAYGSMTWYHGNETGQIPGAFPTKWWEGSALFMSLLLYYYYTGDSTYNDEVRQGMQWQAGDCDYMPSNYSSYLGNDDQMFWGLAAMTAAEIDFADSTDGYSWLALAQGVYNTQVARWDSSNCGGGLRWQIWPYEAGYDMKNSISNGGLFQLAARLARYTNNDTYADWAEKIFDWSASVPLLNNETWNVADSTDIDNGCTTQGNNQWSYNYGTYLMGAAYMYNYTGKAKWKTAVDGLLNVTLTTFFPSKYGGNIMSEELCEPLEVCNDNEILFKGLLSGWLGFVALVVPSTYDQILPKLQGSAEAAAASCSGMSNNTCGVRWYPKSWDGWNGMEEEIAVTNVLSSVLITTKKSGPVTSTTGGNS

SEQ ID NO: 93 - protein, 392 aa, Humicola insolens
DKLQVDLNSPASIKKAAKLVAANLMSHYHGDEPGATPGILPGPPPAGPYYWWQAGAMWGTIVDYWHYTGDETYNAEALRSMVFQAEPPANAYMPRNWTASLGNDDQGFWGMAAMLAAETNFTNPPEDQPQWLALAQAVFNTQVPRWEMDYCNGGLRWQIVQANNGYNYKNTIAAAVFLNIASRLARYTGNDSYAEWAERAWDWMEGVGYITEDFNVKDGAHVESNCTDINPVQFSANAAILIHGVSVMYNYTSGSARDKWRYRVVGLVNRTLEHFFPDGIMVERPCELEDRMQCNTDQHSFKGYMHRALATAAVVAPFMRDTIVPVLRSSTEGCVSSCLADGTCGFRWNIGRYDGDVDHGPAGQQMSALAALSTLLIDQDRVLRGPLTNATG

SEQ ID NO: 94 - protein, 395 aa, Humicola insolens
QQYYKIDTIEEIKESARTLAYDLMLYYKGNQSGEIPGILPGPPTEHKGDYYWWEGGAMMGTYVDYWFLTRDPSYNHVVMEGMLHQVGPNADYMPPNHTASLGNDDQGFWGMSAMLAAENKFPNPPEDQPQWLALAQAVFNTQAAPERHDGTCNGGLRWQVPPTNAGYNYKNTIANACFFDLGARLARYTKNETYANWANNIFDWLMGVGYIDTREPGWRVYDGAHVEHNCTDINKAQFSYNAALLLHGAAFMYNYTNGEEKWKTRIDGLIEGILRDFFKDGAAYELPCEGRQGACTTDMLSFKGYMHRWMAVVTKVAPYTAEKILPALRTSTEAAVAQCTGPPTGRRCGFYWSTRQYVDTAVDKTSGAGEAMNVLAAVSSLLIEYADPPATNETG

SEQ ID NO: 95 - protein, 384 aa, Humicola insolens
AYSIDTVADIKKTAATVAWDLMQYYHGNETGQTPGILPGPPPAGDYYWWEAGAMWGTLIDYWKYTGDDSYNAVITQAMVHQAGPNRDYMPPNVTLSLGNDDQGFWGMSAMLAAELRFPDPPPDQPQWLALAQAVFNTQASPDRHDETCNGGLRWQIPWSNPGYDYKNTIANGCFFNLGARLARYTRNKTYAEWAEKTWDWVEGVGYITKDYQVYDGAHVDHNCTDLNRAQFSYNNAIFLLGAAFMYNYTDGSPKWRDRVEGLVDGAIRDFFPDGVAFEVPCETNMTCTTDMLSFKGYLHRWLAAATTVAPFIAPKVLPVLRSSAEAAISTCTGEADGRTCGFQWAKRQYDGSKGAGQQMNVLGAVSALMVEHNPDYVMVTADSG

SEQ ID NO: 96 - protein, 403 aa, Humicola insolens
IELDLDNEESIKAAASTIAFGLVRYYTGNYTGDTPGNLPDPYFWWEAGAMFGTLVDYWALTGDESYNAITLQAMVHQGTEKGDFMPRNQTRTLGNDDQGFWGMAAMSAAENNFPNPPHDQPQWLALAQSLFNQWASRWEPETCGGGLRWQIFAFNNGFNYKNSISNGCFFNIAARLARYTGNQTYADWAARIWDWEEGIGLITPDYAVHDGVGINPLNGECLVGSMDTNQWTYNAGIFLHGAAVMYNLTNGSADWRARVDGILSNTINTFYTTPEPGSPAGANPVLRELCEGPQNFCNIDQRSFKGYLTRWLAGTSLLAPHTQPVIQPLLRSSALAAAHACSGPTQPPEFKGHTGTACGLRWTTAAGFDGLVGVGEQMNALSAVMYTLAARPGAPEPLTADTG

SEQ ID NO: 97 - protein, 500 aa, Artificial Sequence
  Artificial Sequence
FEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLIS